SEQUENCE LISTING (164 sequences)

SEQ ID NO: 1 - protein, 8 aa, Artificial Sequence
  MSLN-A01 HCDR1
GSIYDFYS

SEQ ID NO: 2 - protein, 8 aa, Artificial Sequence
  MSLN-B03 HCDR1
GFIYRFYV

SEQ ID NO: 3 - protein, 8 aa, Artificial Sequence
  MSLN-A06 HCDR1
GSISGFYA

SEQ ID NO: 4 - protein, 8 aa, Artificial Sequence
  MSLN-H04 HCDR1
GSIFGFYA

SEQ ID NO: 5 - protein, 8 aa, Artificial Sequence
  MSLN-C05 HCDR1
GSTSGIYA

SEQ ID NO: 6 - protein, 8 aa, Artificial Sequence
  MSLN-E05 HCDR1
GSTFSSYY

SEQ ID NO: 7 - protein, 8 aa, Artificial Sequence
  MSLN-D01 HCDR1
GRIFRFYA

SEQ ID NO: 8 - protein, 8 aa, Artificial Sequence
  MSLN-D08 HCDR1
GFTSNYYA

SEQ ID NO: 9 - protein, 8 aa, Artificial Sequence
  MSLN-H02 HCDR1
GSTFSFDF

SEQ ID NO: 10 - protein, 8 aa, Artificial Sequence
  MSLN-A02 HCDR1
GFIFSFYF

SEQ ID NO: 11 - protein, 8 aa, Artificial Sequence
  MSLN-H08 HCDR1
GFIYSFDY

SEQ ID NO: 12 - protein, 8 aa, Artificial Sequence
  MSLN-A03 HCDR1
GFIFNSNL

SEQ ID NO: 13 - protein, 8 aa, Artificial Sequence
  MSLN-A05 HCDR1
GYISNSYV

SEQ ID NO: 14 - protein, 8 aa, Artificial Sequence
  MSLN-F01 HCDR1
GSILNLDV

SEQ ID NO: 15 - protein, 8 aa, Artificial Sequence
  MSLN-G01 HCDR1
GSIYNVNV

SEQ ID NO: 16 - protein, 8 aa, Artificial Sequence
  MSLN-A09 HCDR1
GFIFGHNT

SEQ ID NO: 17 - protein, 8 aa, Artificial Sequence
  MSLN-H06 HCDR1
GFIFNFDY

SEQ ID NO: 18 - protein, 8 aa, Artificial Sequence
  H004-11-41BB HCDR1
GFIYSFDY

SEQ ID NO: 19 - protein, 8 aa, Artificial Sequence
  H004-16-41BB HCDR1
GFIFGHNT

SEQ ID NO: 20 - protein, 8 aa, Artificial Sequence
  H004-3-41BB HCDR1
GSISGFYA

SEQ ID NO: 21 - protein, 8 aa, Artificial Sequence
  H004-4-41BB HCDR1
GSIFGFYA

SEQ ID NO: 22 - protein, 8 aa, Artificial Sequence
  H004BC2-1 HCDR1
GSIYNAYA

SEQ ID NO: 23 - protein, 8 aa, Artificial Sequence
  H004BC2-2 HCDR1
GSTFSYYA

SEQ ID NO: 24 - protein, 8 aa, Artificial Sequence
  H004BC2-3 HCDR1
GFISSFYA

SEQ ID NO: 25 - protein, 8 aa, Artificial Sequence
  H004BC2-4 HCDR1
GYIYGFYA

SEQ ID NO: 26 - protein, 8 aa, Artificial Sequence
  H004BC2-5 HCDR1
GGIFDFYA

SEQ ID NO: 27 - protein, 8 aa, Artificial Sequence
  H004BC2-6 HCDR1
GSISSIYA

SEQ ID NO: 28 - protein, 8 aa, Artificial Sequence
  H004BC2-7 HCDR1
GYTSNFDV

SEQ ID NO: 29 - protein, 8 aa, Artificial Sequence
  H004BC2-8 HCDR1
GSISDLYA

SEQ ID NO: 30 - protein, 8 aa, Artificial Sequence
  H004BC2-9 HCDR1
GFISNFYS

SEQ ID NO: 31 - protein, 8 aa, Artificial Sequence
  H004BC2-10 HCDR1
GGTLRFYA

SEQ ID NO: 32 - protein, 8 aa, Artificial Sequence
  H004BC2-11 HCDR1
GGTLRFYA

SEQ ID NO: 33 - protein, 8 aa, Artificial Sequence
  H004BC2-12 HCDR1
GFTSGIYA

SEQ ID NO: 34 - protein, 8 aa, Artificial Sequence
  H004BC2-13 HCDR1
GFISNFYA

SEQ ID NO: 35 - protein, 8 aa, Artificial Sequence
  H004BC2-14 HCDR1
GFTYDAYA

SEQ ID NO: 36 - protein, 8 aa, Artificial Sequence
  H004BC2-15 HCDR1
GSTSNYYA

SEQ ID NO: 37 - protein, 8 aa, Artificial Sequence
  H004BC2-16 HCDR1
GVTFNFYA

SEQ ID NO: 38 - protein, 8 aa, Artificial Sequence
  H004BC2-17 HCDR1
GSTYGLYA

SEQ ID NO: 39 - protein, 8 aa, Artificial Sequence
  H004BC2-18 HCDR1
GSIYSIYA

SEQ ID NO: 40 - protein, 8 aa, Artificial Sequence
  H004BC2-19 HCDR1
GVTLGFYA

SEQ ID NO: 41 - protein, 8 aa, Artificial Sequence
  H004BC2-20 HCDR1
GFTSNFYS

SEQ ID NO: 42 - protein, 9 aa, Artificial Sequence
  MSLN-A01 HCDR2
TITAFGDIT

SEQ ID NO: 43 - protein, 8 aa, Artificial Sequence
  MSLN-B03 HCDR2
INPFDDNT

SEQ ID NO: 44 - protein, 9 aa, Artificial Sequence
  MSLN-A06 HCDR2
SINSFDSIT

SEQ ID NO: 45 - protein, 8 aa, Artificial Sequence
  MSLN-H04 HCDR2
INSFDGIT

SEQ ID NO: 46 - protein, 8 aa, Artificial Sequence
  MSLN-C05 HCDR2
INSFSDIT

SEQ ID NO: 47 - protein, 8 aa, Artificial Sequence
  MSLN-E05 HCDR2
INSFDDST

SEQ ID NO: 48 - protein, 8 aa, Artificial Sequence
  MSLN-D01 HCDR2
INTIDSIT

SEQ ID NO: 49 - protein, 8 aa, Artificial Sequence
  MSLN-D08 HCDR2
INTLDSIT

SEQ ID NO: 50 - protein, 8 aa, Artificial Sequence
  MSLN-H02 HCDR2
INTHDSIT

SEQ ID NO: 51 - protein, 8 aa, Artificial Sequence
  MSLN-A02 HCDR2
ISRTGGST

SEQ ID NO: 52 - protein, 8 aa, Artificial Sequence
  MSLN-H08 HCDR2
ISRTGGST

SEQ ID NO: 53 - protein, 8 aa, Artificial Sequence
  MSLN-A03 HCDR2
ISRTGGST

SEQ ID NO: 54 - protein, 8 aa, Artificial Sequence
  MSLN-A05 HCDR2
ISRTGGST

SEQ ID NO: 55 - protein, 8 aa, Artificial Sequence
  MSLN-F01 HCDR2
ISRTGGST

SEQ ID NO: 56 - protein, 8 aa, Artificial Sequence
  MSLN-G01 HCDR2
ISRTGGST

SEQ ID NO: 57 - protein, 8 aa, Artificial Sequence
  MSLN-A09 HCDR2
ISRTGGST

SEQ ID NO: 58 - protein, 8 aa, Artificial Sequence
  MSLN-H06 HCDR2
INAYSDST

SEQ ID NO: 59 - protein, 8 aa, Artificial Sequence
  H004-11-41BB HCDR2
ISRTGGST

SEQ ID NO: 60 - protein, 8 aa, Artificial Sequence
  H004-16-41BB HCDR2
ISRTGGST

SEQ ID NO: 61 - protein, 9 aa, Artificial Sequence
  H004-3-41BB HCDR2
SINSFDSIT

SEQ ID NO: 62 - protein, 8 aa, Artificial Sequence
  H004-4-41BB HCDR2
INSFDGIT

SEQ ID NO: 63 - protein, 8 aa, Artificial Sequence
  H004BC2-1 HCDR2
INRIDSTT

SEQ ID NO: 64 - protein, 8 aa, Artificial Sequence
  H004BC2-2 HCDR2
INAFSDIT

SEQ ID NO: 65 - protein, 8 aa, Artificial Sequence
  H004BC2-3 HCDR2
INTFSDIT

SEQ ID NO: 66 - protein, 8 aa, Artificial Sequence
  H004BC2-4 HCDR2
INSIDSIT

SEQ ID NO: 67 - protein, 8 aa, Artificial Sequence
  H004BC2-5 HCDR2
INSFDDIT

SEQ ID NO: 68 - protein, 8 aa, Artificial Sequence
  H004BC2-6 HCDR2
INTVDDIT

SEQ ID NO: 69 - protein, 8 aa, Artificial Sequence
  H004BC2-7 HCDR2
INSIDDIT

SEQ ID NO: 70 - protein, 8 aa, Artificial Sequence
  H004BC2-8 HCDR2
INSFSDIT

SEQ ID NO: 71 - protein, 8 aa, Artificial Sequence
  H004BC2-9 HCDR2
INSFDSIT

SEQ ID NO: 72 - protein, 8 aa, Artificial Sequence
  H004BC2-10 HCDR2
INSYDDIT

SEQ ID NO: 73 - protein, 8 aa, Artificial Sequence
  H004BC2-11 HCDR2
INSYDDIT

SEQ ID NO: 74 - protein, 8 aa, Artificial Sequence
  H004BC2-12 HCDR2
INSFSDIT

SEQ ID NO: 75 - protein, 8 aa, Artificial Sequence
  H004BC2-13 HCDR2
INSYSDIT

SEQ ID NO: 76 - protein, 8 aa, Artificial Sequence
  H004BC2-14 HCDR2
INTFDSNT

SEQ ID NO: 77 - protein, 8 aa, Artificial Sequence
  H004BC2-15 HCDR2
INSFDSIT

SEQ ID NO: 78 - protein, 8 aa, Artificial Sequence
  H004BC2-16 HCDR2
INSYNDIT

SEQ ID NO: 79 - protein, 8 aa, Artificial Sequence
  H004BC2-17 HCDR2
INSFSDIT

SEQ ID NO: 80 - protein, 8 aa, Artificial Sequence
  H004BC2-18 HCDR2
INSYSDIT

SEQ ID NO: 81 - protein, 8 aa, Artificial Sequence
  H004BC2-19 HCDR2
INSYDDIT

SEQ ID NO: 82 - protein, 8 aa, Artificial Sequence
  H004BC2-20 HCDR2
INSFDSIT

SEQ ID NO: 83 - protein, 10 aa, Artificial Sequence
  MSLN-A01 HCDR3
AFFFNFKFSY

SEQ ID NO: 84 - protein, 10 aa, Artificial Sequence
  MSLN-B03 HCDR3
AIWPFGDYGY

SEQ ID NO: 85 - protein, 10 aa, Artificial Sequence
  MSLN-A06 HCDR3
VREKGKGFSY

SEQ ID NO: 86 - protein, 10 aa, Artificial Sequence
  MSLN-H04 HCDR3
VRFRHMGFNY

SEQ ID NO: 87 - protein, 10 aa, Artificial Sequence
  MSLN-C05 HCDR3
VRERGYNFGY

SEQ ID NO: 88 - protein, 10 aa, Artificial Sequence
  MSLN-E05 HCDR3
VRPRWRLFGY

SEQ ID NO: 89 - protein, 10 aa, Artificial Sequence
  MSLN-D01 HCDR3
ARNATSGFSY

SEQ ID NO: 90 - protein, 10 aa, Artificial Sequence
  MSLN-D08 HCDR3
ARNKYQAFGY

SEQ ID NO: 91 - protein, 10 aa, Artificial Sequence
  MSLN-H02 HCDR3
VTVRYKVFSY

SEQ ID NO: 92 - protein, 11 aa, Artificial Sequence
  MSLN-A02 HCDR3
VVFHNWVGFGY

SEQ ID NO: 93 - protein, 10 aa, Artificial Sequence
  MSLN-H08 HCDR3
VRMRWQMFGY

SEQ ID NO: 94 - protein, 13 aa, Artificial Sequence
  MSLN-A03 HCDR3
VIIDKFNYIGFGY

SEQ ID NO: 95 - protein, 10 aa, Artificial Sequence
  MSLN-A05 HCDR3
AMATPDVLDY

SEQ ID NO: 96 - protein, 10 aa, Artificial Sequence
  MSLN-F01 HCDR3
ARHTYISFGY

SEQ ID NO: 97 - protein, 10 aa, Artificial Sequence
  MSLN-G01 HCDR3
VLTTPQSYGY

SEQ ID NO: 98 - protein, 21 aa, Artificial Sequence
  MSLN-A09 HCDR3
ALTRSDRPFTGSYRPEYSFDY

SEQ ID NO: 99 - protein, 10 aa, Artificial Sequence
  MSLN-H06 HCDR3
VKTSYKIYGY

SEQ ID NO: 100 - protein, 11 aa, Artificial Sequence
  H004-11-41BB HCDR3
AVRMRWQMFGY

SEQ ID NO: 101 - protein, 21 aa, Artificial Sequence
  H004-16-41BB HCDR3
ALTRSDRPFTGSYRPEYSFDY

SEQ ID NO: 102 - protein, 10 aa, Artificial Sequence
  H004-3-41BB HCDR3
VREKGKGFSY

SEQ ID NO: 103 - protein, 10 aa, Artificial Sequence
  H004-4-41BB HCDR3
VRFRHMGFNY

SEQ ID NO: 104 - protein, 10 aa, Artificial Sequence
  H004BC2-1 HCDR3
ARYIHAGFDY

SEQ ID NO: 105 - protein, 10 aa, Artificial Sequence
  H004BC2-2 HCDR3
VREIYRSFGY

SEQ ID NO: 106 - protein, 10 aa, Artificial Sequence
  H004BC2-3 HCDR3
VRLYREAFSY

SEQ ID NO: 107 - protein, 10 aa, Artificial Sequence
  H004BC2-4 HCDR3
ARYIAAGFGY

SEQ ID NO: 108 - protein, 10 aa, Artificial Sequence
  H004BC2-5 HCDR3
ARYYRVGFNY

SEQ ID NO: 109 - protein, 10 aa, Artificial Sequence
  H004BC2-6 HCDR3
ARYMTQSFSY

SEQ ID NO: 110 - protein, 10 aa, Artificial Sequence
  H004BC2-7 HCDR3
ATYKWTLFSY

SEQ ID NO: 111 - protein, 10 aa, Artificial Sequence
  H004BC2-8 HCDR3
ARMIKKGFDY

SEQ ID NO: 112 - protein, 10 aa, Artificial Sequence
  H004BC2-9 HCDR3
ARLVRQAFNY

SEQ ID NO: 113 - protein, 10 aa, Artificial Sequence
  H004BC2-10 HCDR3
ARYAFQGFNY

SEQ ID NO: 114 - protein, 10 aa, Artificial Sequence
  H004BC2-11 HCDR3
VRYAFQGFNY

SEQ ID NO: 115 - protein, 10 aa, Artificial Sequence
  H004BC2-12 HCDR3
ARIFMKGFNY

SEQ ID NO: 116 - protein, 10 aa, Artificial Sequence
  H004BC2-13 HCDR3
ARWQGQGFGY

SEQ ID NO: 117 - protein, 10 aa, Artificial Sequence
  H004BC2-14 HCDR3
ARHMGMAFSY

SEQ ID NO: 118 - protein, 10 aa, Artificial Sequence
  H004BC2-15 HCDR3
ARKKGLAFSY

SEQ ID NO: 119 - protein, 10 aa, Artificial Sequence
  H004BC2-16 HCDR3
ARYAFQGFNY

SEQ ID NO: 120 - protein, 10 aa, Artificial Sequence
  H004BC2-17 HCDR3
VRYYSKGFNY

SEQ ID NO: 121 - protein, 10 aa, Artificial Sequence
  H004BC2-18 HCDR3
ARFYQVGFNY

SEQ ID NO: 122 - protein, 10 aa, Artificial Sequence
  H004BC2-19 HCDR3
ARFSLQGFNY

SEQ ID NO: 123 - protein, 10 aa, Artificial Sequence
  H004BC2-20 HCDR3
ARKKGLAFSY

SEQ ID NO: 124 - protein, 118 aa, Artificial Sequence
  MSLN-A01 VH
EVQLVESGGGLVQPGGSLRLSCAASGSIYDFYSMGWFRQAPGKGRELVAATITAFGDITYYPDSVEGRFTISRDNAKRMVYLQMNSLRAEDTAVYYCAFFFNFKFSYWGQGTQVTVSS

SEQ ID NO: 125 - protein, 117 aa, Artificial Sequence
  MSLN-B03 VH
EVQLVESGGGLVQPGGSLRLSCAASGFIYRFYVMGWFRQAPGKGRELVAAINPFDDNTYYPDSVEGRFTISRDNAKRMVYLQMNSLRAEDTAVYYCAIWPFGDYGYWGQGTQVTVSS

SEQ ID NO: 126 - protein, 118 aa, Artificial Sequence
  MSLN-A06 VH
EVQLVESGGGLVQPGGSLRLSCAASGSISGFYAMGWFRQAPGKGRELVAASINSFDSITYYPDSVEGRFTISRDNAKRMVYLQMNSLRAEDTAVYYCVREKGKGFSYWGQGTQVTVSS

SEQ ID NO: 127 - protein, 117 aa, Artificial Sequence
  MSLN-H04 VH
EVQLVESGGGLVQPGGSLRLSCAASGSIFGFYAMGWFRQAPGKGRELVAAINSFDGITYYPDSVEGRFTISRDNAKRMVYLQMNSLRAEDTAVYYCVRFRHMGFNYWGQGTQVTVSS

SEQ ID NO: 128 - protein, 117 aa, Artificial Sequence
  MSLN-C05 VH
EVQLVESGGGLVQPGGSLRLSCAASGSTSGIYAMGWFRQAPGKGRELVAAINSFSDITYYPDSVEGRFTISRDNAKRMVYLQMNSLRAEDTAVYYCVRERGYNFGYWGQGTQVTVSS

SEQ ID NO: 129 - protein, 117 aa, Artificial Sequence
  MSLN-E05 VH
EVQLVESGGGLVQPGGSLRLSCAASGSTFSSYYMGWFRQAPGKGRELVAAINSFDDSTYYPDSVEGRFTISRDNAKRMVYLQMNSLRAEDTAVYYCVRPRWRLFGYWGQGTQVTVSS

SEQ ID NO: 130 - protein, 117 aa, Artificial Sequence
  MSLN-D01 VH
EVQLVESGGGLVQPGGSLRLSCAASGRIFRFYAMGWFRQAPGKGRELVAAINTIDSITYYPDSVEGRFTISRDNAKRMVYLQMNSLRAEDTAVYYCARNATSGFSYWGQGTQVTVSS

SEQ ID NO: 131 - protein, 117 aa, Artificial Sequence
  MSLN-D08 VH
EVQLVESGGGLVQPGGSLRLSCAASGFTSNYYAMGWFRQAPGKGRELVAAINTLDSITYYPDSVEGRFTISRDNAKRMVYLQMNSLRAEDTAVYYCARNKYQAFGYWGQGTQVTVSS

SEQ ID NO: 132 - protein, 117 aa, Artificial Sequence
  MSLN-H02 VH
EVQLVESGGGLVQPGGSLRLSCAASGSTFSFDFMGWFRQAPGKGRELVAAINTHDSITYYPDSVEGRFTISRDNAKRMVYLQMNSLRAEDTAVYYCVTVRYKVFSYWGQGTQVTVSS

SEQ ID NO: 133 - protein, 118 aa, Artificial Sequence
  MSLN-A02 VH
EVQLVESGGGLVQPGGSLRLSCAASGFIFSFYFMGWFRQAPGKGRELVAAISRTGGSTYYPDSVEGRFTISRDNAKRMVYLQMNSLRAEDTAVYYCVVFHNWVGFGYWGQGTQVTVSS

SEQ ID NO: 134 - protein, 117 aa, Artificial Sequence
  MSLN-H08 VH
EVQLVESGGGLVQPGGSLRLSCAASGFIYSFDYMGWFRQAPGKGRELVAAISRTGGSTYYPDSVEGRFTISRDNAKRMVYLQMNSLRAEDTAVYYCVRMRWQMFGYWGQGTQVTVSS

SEQ ID NO: 135 - protein, 120 aa, Artificial Sequence
  MSLN-A03 VH
EVQLVESGGGLVQPGGSLRLSCAASGFIFNSNLMGWFRQAPGKGRELVAAISRTGGSTYYPDSVEGRFTISRDNAKRMVYLQMNSLRAEDTAVYYCVIIDKFNYIGFGYWGQGTQVTVSS

SEQ ID NO: 136 - protein, 117 aa, Artificial Sequence
  MSLN-A05 VH
EVQLVESGGGLVQPGGSLRLSCAASGYISNSYVMGWFRQAPGKGRELVAAISRTGGSTYYPDSVEGRFTISRDNAKRMVYLQMNSLRAEDTAVYYCAMATPDVLDYWGQGTQVTVSS

SEQ ID NO: 137 - protein, 117 aa, Artificial Sequence
  MSLN-F01 VH
EVQLVESGGGLVQPGGSLRLSCAASGSILNLDVMGWFRQAPGKGRELVAAISRTGGSTYYPDSVEGRFTISRDNAKRMVYLQMNSLRAEDTAVYYCARHTYISFGYWGQGTQVTVSS

SEQ ID NO: 138 - protein, 117 aa, Artificial Sequence
  MSLN-G01 VH
EVQLVESGGGLVQPGGSLRLSCAASGSIYNVNVMGWFRQAPGKGRELVAAISRTGGSTYYPDSVEGRFTISRDNAKRMVYLQMNSLRAEDTAVYYCVLTTPQSYGYWGQGTQVTVSS

SEQ ID NO: 139 - protein, 128 aa, Artificial Sequence
  MSLN-A09 VH
EVQLVESGGGLVQPGGSLRLSCAASGFIFGHNTMGWFRQAPGKGRELVAAISRTGGSTYYPDSVEGRFTISRDNAKRMVYLQMNSLRAEDTAVYYCALTRSDRPFTGSYRPEYSFDYWGQGTQVTVSS

SEQ ID NO: 140 - protein, 117 aa, Artificial Sequence
  MSLN-H06 VH
EVQLVESGGGLVQPGGSLRLSCAASGFIFNFDYMGWFRQAPGKGRELVAAINAYSDSTYYPDSVEGRFTISRDNAKRMVYLQMNSLRAEDTAVYYCVKTSYKIYGYWGQGTQVTVSS

SEQ ID NO: 141 - protein, 118 aa, Artificial Sequence
  H004-11-41BB VH
EVQLVESGGGLVQPGGSLRLSCAASGFIYSFDYMGWFRQAPGKGRELVAAISRTGGSTYYPDSVEGRFTISRDNAKRMVYLQMNSLRAEDTAVYYCAVRMRWQMFGYWGQGTQVTVSS

SEQ ID NO: 142 - protein, 128 aa, Artificial Sequence; identical to SEQ ID NO: 139
  H004-16-41BB VH

SEQ ID NO: 143 - protein, 118 aa, Artificial Sequence; identical to SEQ ID NO: 126
  H004-3-41BB VH

SEQ ID NO: 144 - protein, 117 aa, Artificial Sequence; identical to SEQ ID NO: 127
  H004-4-41BB VH

SEQ ID NO: 145 - protein, 118 aa, Artificial Sequence
  H004BC2-1 VH
EVQLVESGGGLVQPGGSLRLSCAASGSIYNAYAMGWFRQAPGKGRELVATINRIDSTTYYPDSVEGRFTISRDNAKRMVYLQMNSLRAEDTAVYYCAARYIHAGFDYWGQGTQVTVSS

SEQ ID NO: 146 - protein, 118 aa, Artificial Sequence
  H004BC2-2 VH
EVQLVESGGGLVQPGGSLRLSCAASGSTFSYYAMGWFRQAPGKGRELVATINAFSDITYYPDSVEGRFTISRDNAKRMVYLQMNSLRAEDTAVYYCAVREIYRSFGYWGQGTQVTVSS

SEQ ID NO: 147 - protein, 118 aa, Artificial Sequence
  H004BC2-3 VH
EVQLVESGGGLVQPGGSLRLSCAASGFISSFYAMGWFRQAPGKGRELVATINTFSDITYYPDSVEGRFTISRDNAKRMVYLQMNSLRAEDTAVYYCAVRLYREAFSYWGQGTQVTVSS

SEQ ID NO: 148 - protein, 118 aa, Artificial Sequence
  H004BC2-4 VH
EVQLVESGGGLVQPGGSLRLSCAASGYIYGFYAMGWFRQAPGKGRELVASINSIDSITYYPDSVEGRFTISRDNAKRMVYLQMNSLRAEDTAVYYCAARYIAAGFGYWGQGTQVTVSS

SEQ ID NO: 149 - protein, 118 aa, Artificial Sequence
  H004BC2-5 VH
EVQLVESGGGLVQPGGSLRLSCAASGGIFDFYAMGWFRQAPGKGRELVASINSFDDITYYPDSVEGRFTISRDNAKRMVYLQMNSLRAEDTAVYYCAARYYRVGFNYWGQGTQVTVSS

SEQ ID NO: 150 - protein, 118 aa, Artificial Sequence
  H004BC2-6 VH
EVQLVESGGGLVQPGGSLRLSCAASGSISSIYAMGWFRQAPGKGRELVATINTVDDITYYPDSVEGRFTISRDNAKRMVYLQMNSLRAEDTAVYYCAARYMTQSFSYWGQGTQVTVSS

SEQ ID NO: 151 - protein, 118 aa, Artificial Sequence
  H004BC2-7 VH
EVQLVESGGGLVQPGGSLRLSCAASGYTSNFDVMGWFRQAPGKGRELVAAINSIDDITYYPDSVEGRFTISRDNAKRMVYLQMNSLRAEDTAVYYCAATYKWTLFSYWGQGTQVTVSS

SEQ ID NO: 152 - protein, 118 aa, Artificial Sequence
  H004BC2-8 VH
EVQLVESGGGLVQPGGSLRLSCAASGSISDLYAMGWFRQAPGKGRELVASINSFSDITYYPDSVEGRFTISRDNAKRMVYLQMNSLRAEDTAVYYCAARMIKKGFDYWGQGTQVTVSS

SEQ ID NO: 153 - protein, 118 aa, Artificial Sequence
  H004BC2-9 VH
EVQLVESGGGLVQPGGSLRLSCAASGFISNFYSMGWFRQAPGKGRELVASINSFDSITYYPDSVEGRFTISRDNAKRMVYLQMNSLRAEDTAVYYCAARLVRQAFNYWGQGTQVTVSS

SEQ ID NO: 154 - protein, 118 aa, Artificial Sequence
  H004BC2-10 VH
EVQLVESGGGLVQPGGSLRLSCAASGGTLRFYAMGWFRQAPGKGRELVATINSYDDITYYPDSVEGRFTISRDNAKRMVYLQMNSLRAEDTAVYYCAARYAFQGFNYWGQGTQVTVSS

SEQ ID NO: 155 - protein, 118 aa, Artificial Sequence
  H004BC2-11 VH
EVQLVESGGGLVQPGGSLRLSCAASGGTLRFYAMGWFRQAPGKGRELVATINSYDDITYYPDSVEGRFTISRDNAKRMVYLQMNSLRAEDTAVYYCAVRYAFQGFNYWGQGTQVTVSS

SEQ ID NO: 156 - protein, 118 aa, Artificial Sequence
  H004BC2-12 VH
EVQLVESGGGLVQPGGSLRLSCAASGFTSGIYAMGWFRQAPGKGRELVATINSFSDITYYPDSVEGRFTISRDNAKRMVYLQMNSLRAEDTAVYYCAARIFMKGFNYWGQGTQVTVSS

SEQ ID NO: 157 - protein, 118 aa, Artificial Sequence
  H004BC2-13 VH
EVQLVESGGGLVQPGGSLRLSCAASGFISNFYAMGWFRQAPGKGRELVASINSYSDITYYPDSVEGRFTISRDNAKRMVYLQMNSLRAEDTAVYYCAARWQGQGFGYWGQGTQVTVSS

SEQ ID NO: 158 - protein, 118 aa, Artificial Sequence
  H004BC2-14 VH
EVQLVESGGGLVQPGGSLRLSCAASGFTYDAYAMGWFRQAPGKGRELVATINTFDSNTYYPDSVEGRFTISRDNAKRMVYLQMNSLRAEDTAVYYCAARHMGMAFSYWGQGTQVTVSS

SEQ ID NO: 159 - protein, 118 aa, Artificial Sequence
  H004BC2-15 VH
EVQLVESGGGLVQPGGSLRLSCAASGSTSNYYAMGWFRQAPGKGRELVASINSFDSITYYPDSVEGRFTISRDNAKRMVYLQMNSLRAEDTAVYYCAARKKGLAFSYWGQGTQVTVSS

SEQ ID NO: 160 - protein, 118 aa, Artificial Sequence
  H004BC2-16 VH
EVQLVESGGGLVQPGGSLRLSCAASGVTFNFYAMGWFRQAPGKGRELVATINSYNDITYYPDSVEGRFTISRDNAKRMVYLQMNSLRAEDTAVYYCAARYAFQGFNYWGQGTQVTVSS

SEQ ID NO: 161 - protein, 118 aa, Artificial Sequence
  H004BC2-17 VH
EVQLVESGGGLVQPGGSLRLSCAASGSTYGLYAMGWFRQAPGKGRELVASINSFSDITYYPDSVEGRFTISRDNAKRMVYLQMNSLRAEDTAVYYCAVRYYSKGFNYWGQGTQVTVSS

SEQ ID NO: 162 - protein, 118 aa, Artificial Sequence
  H004BC2-18 VH
EVQLVESGGGLVQPGGSLRLSCAASGSIYSIYAMGWFRQAPGKGRELVATINSYSDITYYPDSVEGRFTISRDNAKRMVYLQMNSLRAEDTAVYYCAARFYQVGFNYWGQGTQVTVSS

SEQ ID NO: 163 - protein, 118 aa, Artificial Sequence
  H004BC2-19 VH
EVQLVESGGGLVQPGGSLRLSCAASGVTLGFYAMGWFRQAPGKGRELVATINSYDDITYYPDSVEGRFTISRDNAKRMVYLQMNSLRAEDTAVYYCAARFSLQGFNYWGQGTQVTVSS

SEQ ID NO: 164 - protein, 118 aa, Artificial Sequence
  H004BC2-20 VH
EVQLVESGGGLVQPGGSLRLSCAASGFTSNFYSMGWFRQAPGKGRELVASINSFDSITYYPDSVEGRFTISRDNAKRMVYLQMNSLRAEDTAVYYCAARKKGLAFSYWGQGTQVTVSS

(19) United States
(12) Patent Application Publication
(10) Pub. No.: US 2024/0050569 A1
(43) Pub. Date: Feb. 15, 2024

(54) MESOTHELIN BINDING MOLECULES AND USES THEREOF

(71) Applicant: BioArdis, LLC, San Diego, CA (US)

(72) Inventors: Farzad Haerizadeh, San Diego, CA (US); Masood Tayebi, San Diego, CA (US)

(21) Appl. No.: 18/268,215

(22) PCT Filed: Dec. 16, 2021
(86) PCT No.: PCT/US2021/063857

§ 371 Date: Jun. 16, 2023

Related U.S. Application Data
(60) Provisional application No. 63/127,885, filed on Dec. 18, 2020.
(60) Provisional application No. 63/262,309, filed on Oct. 8, 2021.

(51) Int. Cl. A61K 39/00; C07K 16/30; A61P 35/00; C07K 16/28

(52) U.S. Cl. CPC A61K 39/464468; C07K 16/30; A61K 39/4611; A61K 39/4631; A61P 35/00; C07K 16/2866; C07K 2317/24; C07K 2317/569

(57) ABSTRACT

Disclosed herein are mesothelin (MSLN)-specific binding polypeptides. These binding polypeptides may be incorporated into chimeric antigen receptors (CARs). Also disclosed herein are methods of using these binding polypeptides and/or CARs for the treatment of, for example, a cancer.

### CROSS-REFERENCE TO RELATED APPLICATIONS

This application claims the benefit of priority of U.S. Provisional Patent Application No. 63/127,885, filed Dec. 18, 2020, and U.S. Provisional Patent Application No. 63/262,309, filed Oct. 8, 2021, each of which is hereby expressly incorporated by reference in its entirety.

### REFERENCE TO SEQUENCE LISTING

The present application is being filed along with a Sequence Listing in electronic format. The Sequence Listing is provided as a file entitled BWGB006_SeqListing.TXT, which was created and last modified on Dec. 16, 2021, which is 69,451 bytes in size. The information in the electronic Sequence Listing is hereby incorporated by reference in its entirety.

### FIELD

Aspects of the present disclosure relate generally to mesothelin (MSLN)-specific binding polypeptides. These binding polypeptides can be incorporated into chimeric antigen receptor (CAR) constructs to be expressed in immune cells. These binding polypeptides and CARs may be used in the treatment of cancer.

### BACKGROUND

Chimeric antigen receptor (CAR) T cells and other adoptive cell therapies have been shown to be effective in the treatment of cancer. The CAR, which is made up of an extracellular antigen binding domain, a transmembrane domain, and an intracellular signaling domain, enables directed killing of cancer cells based on cell surface antigen expression while minimally affecting normal cells that are not expressing the targeted antigen. The extracellular antigen binding domain is often made up of an antibody or a binding fragment or derivative thereof, such as a single chain variable fragment (scFv) or single domain antibody (sdAb). There is a present need for improved extracellular antigen binding domains to be used in CARs for the treatment of various cancers or other diseases.

### SUMMARY OF THE DISCLOSURE

Disclosed herein are binding polypeptides that are able to bind to mesothelin (MSLN). These binding polypeptides may be incorporated in a chimeric antigen receptor (CAR), which can be expressed by a cell. In some embodiments, the binding polypeptides are single domain antibodies (sdAbs).

Disclosed herein in some embodiments are MSLN binding polypeptides comprising an immunoglobulin heavy chain variable domain comprising a CDR-H1, CDR-H2, and CDR-H3. In some embodiments, the CDR-H1 comprises a sequence having at least 90%, 95%, 99%, or 100% sequence identity to a sequence selected from SEQ ID NOs: 1-41. In some embodiments, CDR-H2 comprises a sequence having at least 90%, 95%, 99%, or 100% sequence identity to a sequence selected from SEQ ID NOs: 42-82. In some embodiments, the CDR-H3 comprises a sequence having at least 90%, 95%, 99%, or 100% sequence identity to a sequence selected from SEQ ID NOs: 83-123. In some embodiments, the CDR-H1 comprises a sequence having at least 90%, 95%, 99%, or 100% sequence identity to a sequence selected from SEQ ID NOs: 1-41, the CDR-H2 comprises a sequence having at least 90%, 95%, 99%, or 100% sequence identity to a sequence selected from SEQ ID NOs: 42-82, and the CDR-H3 comprises a sequence having at least 90%, 95%, 99%, or 100% sequence identity to a sequence selected from SEQ ID NOs: 83-123. In some embodiments, the immunoglobulin heavy chain variable domain comprises a sequence having at least 90%, 95%, 99%, or 100% sequence identity to a sequence selected from SEQ ID NOs: 124-164.

Also disclosed herein are nucleic acids that encode for any one of the MSLN binding polypeptides disclosed herein.

Also disclosed herein are methods of treating a cancer in a subject in need thereof. In some embodiments, the methods comprise administering a chimeric antigen receptor cell to the subject. In some embodiments, the chimeric antigen receptor cell is any one of the chimeric antigen receptor cells disclosed herein. In some embodiments, the chimeric antigen receptor cell comprises any one or more of the MSLN binding polypeptides disclosed herein.

Embodiments of the present invention provided herein are described by way of the following numbered alternatives:

1. A mesothelin (MSLN) binding polypeptide comprising an immunoglobulin heavy chain variable domain comprising a CDR-H1, CDR-H2, and CDR-H3, wherein:
the CDR-H1 comprises a sequence having at least 90%, 95%, 99%, or 100% sequence identity to a sequence selected from SEQ ID NOs: 1-41;
the CDR-H2 comprises a sequence having at least 90%, 95%, 99%, or 100% sequence identity to a sequence selected from SEQ ID NOs: 42-82; and
the CDR-H3 comprises a sequence having at least 90%, 95%, 99%, or 100% sequence identity to a sequence selected from SEQ ID NOs: 83-123.

2. The MSLN binding polypeptide of alternative 1, wherein:
1) the CDR-H1 comprises the sequence of SEQ ID NO: 1, the CDR-H2 comprises the sequence of SEQ ID NO: 42, and the CDR-H3 comprises the sequence of SEQ ID NO: 83;
2) the CDR-H1 comprises the sequence of SEQ ID NO: 2, the CDR-H2 comprises the sequence of SEQ ID NO: 43, and the CDR-H3 comprises the sequence of SEQ ID NO: 84;
3) the CDR-H1 comprises the sequence of SEQ ID NO: 3, the CDR-H2 comprises the sequence of SEQ ID NO: 44, and the CDR-H3 comprises the sequence of SEQ ID NO: 85;
4) the CDR-H1 comprises the sequence of SEQ ID NO: 4, the CDR-H2 comprises the sequence of SEQ ID NO: 45, and the CDR-H3 comprises the sequence of SEQ ID NO: 86;
5) the CDR-H1 comprises the sequence of SEQ ID NO: 5, the CDR-H2 comprises the sequence of SEQ ID NO: 46, and the CDR-H3 comprises the sequence of SEQ ID NO: 87;
6) the CDR-H1 comprises the sequence of SEQ ID NO: 6, the CDR-H2 comprises the sequence of SEQ ID NO: 47, and the CDR-H3 comprises the sequence of SEQ ID NO: 88;
7) the CDR-H1 comprises the sequence of SEQ ID NO: 7, the CDR-H2 comprises the sequence of SEQ ID NO: 48, and the CDR-H3 comprises the sequence of SEQ ID NO: 89;
8) the CDR-H1 comprises the sequence of SEQ ID NO: 8, the CDR-H2 comprises the sequence of SEQ ID NO: 49, and the CDR-H3 comprises the sequence of SEQ ID NO: 90;
9) the CDR-H1 comprises the sequence of SEQ ID NO: 9, the CDR-H2 comprises the sequence of SEQ ID NO: 50, and the CDR-H3 comprises the sequence of SEQ ID NO: 91;
10) the CDR-H1 comprises the sequence of SEQ ID NO: 10, the CDR-H2 comprises the sequence of SEQ ID NO: 51, and the CDR-H3 comprises the sequence of SEQ ID NO: 92;
11) the CDR-H1 comprises the sequence of SEQ ID NO: 11, the CDR-H2 comprises the sequence of SEQ ID NO: 52, and the CDR-H3 comprises the sequence of SEQ ID NO: 93;
12) the CDR-H1 comprises the sequence of SEQ ID NO: 12, the CDR-H2 comprises the sequence of SEQ ID NO: 53, and the CDR-H3 comprises the sequence of SEQ ID NO: 94;
13) the CDR-H1 comprises the sequence of SEQ ID NO: 13, the CDR-H2 comprises the sequence of SEQ ID NO: 54, and the CDR-H3 comprises the sequence of SEQ ID NO: 95;
14) the CDR-H1 comprises the sequence of SEQ ID NO: 14, the CDR-H2 comprises the sequence of SEQ ID NO: 55, and the CDR-H3 comprises the sequence of SEQ ID NO: 96;
15) the CDR-H1 comprises the sequence of SEQ ID NO: 15, the CDR-H2 comprises the sequence of SEQ ID NO: 56, and the CDR-H3 comprises the sequence of SEQ ID NO: 97;
16) the CDR-H1 comprises the sequence of SEQ ID NO: 16, the CDR-H2 comprises the sequence of SEQ ID NO: 57, and the CDR-H3 comprises the sequence of SEQ ID NO: 98;
17) the CDR-H1 comprises the sequence of SEQ ID NO: 17, the CDR-H2 comprises the sequence of SEQ ID NO: 58, and the CDR-H3 comprises the sequence of SEQ ID NO: 99;
18) the CDR-H1 comprises the sequence of SEQ ID NO: 18, the CDR-H2 comprises the sequence of SEQ ID NO: 59, and the CDR-H3 comprises the sequence of SEQ ID NO: 100;
19) the CDR-H1 comprises the sequence of SEQ ID NO: 19, the CDR-H2 comprises the sequence of SEQ ID NO: 60, and the CDR-H3 comprises the sequence of SEQ ID NO: 101;
20) the CDR-H1 comprises the sequence of SEQ ID NO: 20, the CDR-H2 comprises the sequence of SEQ ID NO: 61, and the CDR-H3 comprises the sequence of SEQ ID NO: 102;
21) the CDR-H1 comprises the sequence of SEQ ID NO: 21, the CDR-H2 comprises the sequence of SEQ ID NO: 62, and the CDR-H3 comprises the sequence of SEQ ID NO: 103;
22) the CDR-H1 comprises the sequence of SEQ ID NO: 22, the CDR-H2 comprises the sequence of SEQ ID NO: 63, and the CDR-H3 comprises the sequence of SEQ ID NO: 104;
23) the CDR-H1 comprises the sequence of SEQ ID NO: 23, the CDR-H2 comprises the sequence of SEQ ID NO: 64, and the CDR-H3 comprises the sequence of SEQ ID NO: 105;
24) the CDR-H1 comprises the sequence of SEQ ID NO: 24, the CDR-H2 comprises the sequence of SEQ ID NO: 65, and the CDR-H3 comprises the sequence of SEQ ID NO: 106;
25) the CDR-H1 comprises the sequence of SEQ ID NO: 25, the CDR-H2 comprises the sequence of SEQ ID NO: 66, and the CDR-H3 comprises the sequence of SEQ ID NO: 107;
26) the CDR-H1 comprises the sequence of SEQ ID NO: 26, the CDR-H2 comprises the sequence of SEQ ID NO: 67, and the CDR-H3 comprises the sequence of SEQ ID NO: 108;
27) the CDR-H1 comprises the sequence of SEQ ID NO: 27, the CDR-H2 comprises the sequence of SEQ ID NO: 68, and the CDR-H3 comprises the sequence of SEQ ID NO: 109;
28) the CDR-H1 comprises the sequence of SEQ ID NO: 28, the CDR-H2 comprises the sequence of SEQ ID NO: 69, and the CDR-H3 comprises the sequence of SEQ ID NO: 110;
29) the CDR-H1 comprises the sequence of SEQ ID NO: 29, the CDR-H2 comprises the sequence of SEQ ID NO: 70, and the CDR-H3 comprises the sequence of SEQ ID NO: 111;
30) the CDR-H1 comprises the sequence of SEQ ID NO: 30, the CDR-H2 comprises the sequence of SEQ ID NO: 71, and the CDR-H3 comprises the sequence of SEQ ID NO: 112;
31) the CDR-H1 comprises the sequence of SEQ ID NO: 31, the CDR-H2 comprises the sequence of SEQ ID NO: 72, and the CDR-H3 comprises the sequence of SEQ ID NO: 113;
32) the CDR-H1 comprises the sequence of SEQ ID NO: 32, the CDR-H2 comprises the sequence of SEQ ID NO: 73, and the CDR-H3 comprises the sequence of SEQ ID NO: 114;
33) the CDR-H1 comprises the sequence of SEQ ID NO: 33, the CDR-H2 comprises the sequence of SEQ ID NO: 74, and the CDR-H3 comprises the sequence of SEQ ID NO: 115;
34) the CDR-H1 comprises the sequence of SEQ ID NO: 34, the CDR-H2 comprises the sequence of SEQ ID NO: 75, and the CDR-H3 comprises the sequence of SEQ ID NO: 116;
35) the CDR-H1 comprises the sequence of SEQ ID NO: 35, the CDR-H2 comprises the sequence of SEQ ID NO: 76, and the CDR-H3 comprises the sequence of SEQ ID NO: 117;
36) the CDR-H1 comprises the sequence of SEQ ID NO: 36, the CDR-H2 comprises the sequence of SEQ ID NO: 77, and the CDR-H3 comprises the sequence of SEQ ID NO: 118;
37) the CDR-H1 comprises the sequence of SEQ ID NO: 37, the CDR-H2 comprises the sequence of SEQ ID NO: 78, and the CDR-H3 comprises the sequence of SEQ ID NO: 119;
38) the CDR-H1 comprises the sequence of SEQ ID NO: 38, the CDR-H2 comprises the sequence of SEQ ID NO: 79, and the CDR-H3 comprises the sequence of SEQ ID NO: 120;
39) the CDR-H1 comprises the sequence of SEQ ID NO: 39, the CDR-H2 comprises the sequence of SEQ ID NO: 80, and the CDR-H3 comprises the sequence of SEQ ID NO: 121;
40) the CDR-H1 comprises the sequence of SEQ ID NO: 40, the CDR-H2 comprises the sequence of SEQ ID NO: 81, and the CDR-H3 comprises the sequence of SEQ ID NO: 122; or
41) the CDR-H1 comprises the sequence of SEQ ID NO: 41, the CDR-H2 comprises the sequence of SEQ ID NO: 82, and the CDR-H3 comprises the sequence of SEQ ID NO: 123.

3. The MSLN binding polypeptide of alternative 1 or 2, wherein the heavy chain variable domain comprises an amino acid sequence having at least 90%, 95%, 99%, or 100% sequence identity to any sequence selected from SEQ ID NOs: 124-164.

4. The MSLN binding polypeptide of any one of alternatives 1-3, wherein the MSLN binding polypeptide is humanized.

5. The MSLN binding polypeptide of any one of alternatives 1-4, wherein the MSLN binding polypeptide is a single domain antibody (sdAb).

6. A chimeric antigen receptor (CAR) comprising the MSLN binding polypeptide of any one of alternatives 1-5.

7. A chimeric antigen receptor (CAR) cell comprising the CAR of alternative 6.

8. The CAR cell of alternative 7, wherein the CAR cell is a CART cell.

9. The CAR cell of alternative 7 or 8, wherein the CAR cell comprises at least two binding polypeptides and the CAR cell is a multivalent CAR cell.

10. The CAR cell of any one of alternatives 7-9, wherein the CAR cell is derived from a subject or from a cell line.

11. The CAR cell of alternative 10, wherein the subject has a cancer.

12. The CAR cell of alternative 11, wherein the cancer is breast cancer, colorectal cancer, kidney cancer, liver cancer, lung cancer, brain cancer, pancreatic cancer, bladder cancer, testicular cancer, prostate cancer, gastric cancer, ovarian cancer, head and neck cancer, gallbladder cancer, a hematologic malignancy, or any combination thereof.

13. A nucleic acid that encodes for a polypeptide comprising a sequence having at least 90%, 95%, 99%, or 100% sequence identity to the MSLN binding polypeptide of any one of alternatives 1-5 or the CAR of alternative 6.

14. A method of treating a cancer in a subject in need thereof, comprising administering the CAR cell of any one of alternatives 7-12.

15. The method of alternative 14, wherein the chimeric antigen receptor cell is autologous or allogeneic to the subject.

16. The method of alternative 14 or 15, wherein the subject is a mammal.

17. The method of any one of alternatives 14-16, wherein the subject is a human.

18. The method of any one of alternatives 14-17, wherein the cancer is breast cancer, colorectal cancer, kidney cancer, liver cancer, lung cancer, brain cancer, pancreatic cancer, bladder cancer, testicular cancer, prostate cancer, gastric cancer, ovarian cancer, head and neck cancer, gallbladder cancer, a hematologic malignancy, or any combination thereof.

19. The method of any one of alternatives 14-18, wherein the chimeric antigen receptor cell is administered parenterally.

### BRIEF DESCRIPTION OF THE DRAWINGS

In addition to the features described above, additional features and variations will be readily apparent from the following descriptions of the drawings and exemplary embodiments. It is to be understood that these drawings depict typical embodiments and are not intended to be limiting in scope.

FIG. 1 depicts an exemplary alignment for the heavy chain variable domain CDRs disclosed herein.

FIG. 2A depicts a bar chart which shows the results of a cytotoxicity assay of exemplary anti-MSLN CAR T-cell lines against BxPC-3 pancreatic cancer cells.

FIG. 2B depicts a bar chart which shows the results of a cytotoxicity assay of exemplary anti-MSLN CAR T-cell lines against Capan-1 pancreatic cancer cells.

FIG. 2C depicts a bar chart which shows the results of a cytotoxicity assay of exemplary anti-MSLN CAR T-cell lines against NCI-N87 gastric cancer cells, where the T-cells have been obtained from a donor denoted Z0016.

FIG. 2D depicts a bar chart which shows the results of a cytotoxicity assay of exemplary anti-MSLN CAR T-cell lines against NCI-N87 gastric cancer cells, where the T-cell have been obtained from a donor denoted Y1287.

FIG. 3 depicts three line graphs which show the anti-tumor efficacy of an exemplary anti-MSLN CAR T-cell line B4T2-002 ('002) against HCC1806 breast cancer, H2110 non-small cell lung cancer, and NCI-N87 gastric cancer xenograph models.

### DETAILED DESCRIPTION OF THE DISCLOSURE

Disclosed herein are binding polypeptides that are incorporated into a chimeric antigen receptor cell. In some embodiments, the chimeric antigen receptor cell is a chimeric antigen receptor T cell (CAR-T cell). These CAR-Ts may be constructed through processes conventionally known in the art. The binding polypeptides provide specificity towards their respective tumor-associated antigens, enabling targeting of cancers expressing said tumor-associated antigens by the CAR-T cell.

In some embodiments, the binding polypeptides are single domain antibodies (sdAbs) disposed on the surface of the chimeric antigen receptor cells (e.g. CAR-T cell). The sdAbs may be specific for, or have binding affinity towards, a tumor-associated antigen. In some embodiments, the tumor-associated antigen is mesothelin (MSLN).

Also disclosed herein are methods of treating a cancer in a subject in need thereof by administering a chimeric antigen receptor cell comprising one or more of the binding polypeptides disclosed herein. In some embodiments, the cancer may be breast cancer, colorectal cancer, kidney cancer, liver cancer, lung cancer, brain cancer, pancreatic cancer, bladder cancer, testicular cancer, prostate cancer, gastric cancer, ovarian cancer, head and neck cancer, gallbladder cancer, a hematologic malignancy, or any combination thereof. In some embodiments, the hematologic malignancy may comprise leukemia, acute lymphoblastic leukemia, acute myelogenous leukemia, chronic myelogenous leukemia, chronic lymphocytic leukemia, hairy cell leukemia, lymphoma, Hodgkin's disease, Non-Hodgkin lymphoma, or multiple myeloma. The CAR-T cell may be derived from the subject for an autologous treatment. Alternatively, the CAR-T cell may be derived from the same species as the subject for an allogeneic treatment.

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed.

The section headings used herein are for organizational purposes only and are not to be construed as limiting the subject matter described.

The articles “a” and “an” are used herein to refer to one or to more than one (for example, at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

The term “administering” includes enteral, oral, intranasal, parenteral, intravenous, intraperitoneal, intramuscular, intra-arteriole, intraventricular, intradermal, intralesional, intracranial, intrathecal, or subcutaneous administration, or the implantation of a slow-release device, e.g., a mini-osmotic pump, to a subject.

The terms “nucleic acid” or “nucleic acid molecule” as used herein refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Nucleic acid monomers can be linked by phosphodiester bonds or analogs of such linkages. A nucleic acid or nucleic acids can be contained in a nucleic acid vector or nucleic acid construct (e.g. plasmid, virus, bacteriophage, cosmid, fosmid, phagemid, bacterial artificial chromosome (BAC), yeast artificial chromosome (YAC), or human artificial chromosome (HAC)) that can be used for amplification and/or expression of the nucleic acid or nucleic acids in various biological systems. Typically, the vector or construct will also contain elements including but not limited to promoters, enhancers, terminators, inducers, ribosome binding sites, translation initiation sites, start codons, stop codons, polyadenylation signals, origins of replication, cloning sites, multiple cloning sites, restriction enzyme sites, epitopes, reporter genes, selection markers, antibiotic selection markers, targeting sequences, peptide purification tags, or accessory genes, or any combination thereof.

A nucleic acid or nucleic acid molecule can comprise one or more sequences encoding different peptides, polypeptides, or proteins. These one or more sequences can be joined in the same nucleic acid or nucleic acid molecule adjacently, or with extra nucleic acids in between, e.g. linkers, repeats or restriction enzyme sites, or any other sequence that is 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a nucleic acid as used herein refers to a sequence being after the 3′-end of a previous sequence, on the strand containing the encoding sequence (sense strand) if the nucleic acid is double stranded. The term “upstream” on a nucleic acid as used herein refers to a sequence being before the 5′-end of a subsequent sequence, on the strand containing the encoding sequence (sense strand) if the nucleic acid is double stranded. The term “grouped” on a nucleic acid as used herein refers to two or more sequences that occur in proximity either directly or with extra nucleic acids in between, e.g. linkers, repeats, or restriction enzyme sites, or any other sequence that is 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths, but generally not with a sequence in between that encodes for a functioning or catalytic polypeptide, protein, or protein domain.

The term “codon optimized” regarding a nucleic acid as used herein refers to the substitution of codons of the nucleic acid to enhance or maximize translation in a host of a particular species without changing the polypeptide sequence based on species-specific codon usage biases and relative availability of each aminoacyl-tRNA in the target cell cytoplasm. Codon optimization and techniques to perform such optimization is known in the art. Those skilled in the art will appreciate that gene expression levels are dependent on many factors, such as promoter sequences and regulatory elements. In this aspect, many synthetic genes can be designed to increase their protein expression level.

The terms “peptide”, “polypeptide”, and “protein” as used herein refers to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a polypeptide as used herein refers to a sequence being after the C-terminus of a previous sequence. The term “upstream” on a polypeptide as used herein refers to a sequence being before the N-terminus of a subsequent sequence.

In some embodiments, the nucleic acid or peptide sequences presented herein and used in the examples are functional in various biological systems including but not limited to humans, mice, rats, monkeys, primates, cats, dogs, rabbits, E. coli, yeast, and mammalian cells. In other embodiments, nucleic acid or peptide sequences sharing at least or lower than 0%, 10%, 20%, 30%, 40%, 50%, 60%, 70%, 80%, 90%, 91%, 92%, 93%, 94%, 95%, 96%, 97%, 98%, 99%, or 100% identity, or any percentage within a range defined by any two of the aforementioned percentages of identity to the nucleic acid or peptide sequences presented herein and used in the examples can also be used with little or no effect on the function of the sequences in biological systems. As used herein, the term “identity” refers to a nucleic acid or peptide sequence having the same overall order of nucleotide or amino acids, respectively, as a template nucleic acid or peptide sequence with specific changes such as substitutions, deletions, repetitions, or insertions within the sequence. In some embodiments, two nucleic acid sequences sharing as low as 0%, 10%, 20%, 30%, 40%, 50%, 60%, 70%, 80%, 90%, 91%, 92%, 93%, 94%, 95%, 96%, 97%, 98%, or 99% identity can encode for the same polypeptide by comprising different codons that encode for the same amino acid during translation.

As disclosed herein, sequences having a % homology to any of the sequences disclosed herein are envisioned and may be used. The term “% homology” refers to the degree of conservation between two sequences when considering their three-dimensional structure. For example, homology between two protein sequences may be dependent on structural motifs, such as beta strands, alpha helices, and other folds, as well as their distribution throughout the sequence. Homology may be determined through structural determination, either empirically or in silico. In some embodiments, any sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence homology to any of the sequences disclosed herein may be used. In some embodiments, any sequence having at least 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 substitutions, deletions, or additions relative to any of the sequences disclosed herein, which may or may not affect the overall % homology, may be used.

As applied herein, sequences having a certain % similarity to any of the sequence disclosed herein are envisioned and may be used. In some embodiments, these sequences may include peptide sequences, nucleic acid sequences, CDR sequences, variable region sequences, or heavy or light chain sequences. As understood in the art with respect to peptide sequences, “similarity” refers to the comparison of amino acids based on their properties, including but not limited to size, polarity, charge, pK, aromaticity, hydrogen bonding properties, or presence of functional groups (e.g. hydroxyl, thiol, amine, carboxyl, and the like). The term “% similarity” refers to the percentage of units (i.e. amino acids) that are the same between two or more sequences relative to the length of the sequence. When the two or more sequences being compared are the same length, the % similarity will be respective that length. When two or more sequences being compared are different lengths, deletions and/or insertions may be introduced to obtain the best alignment. The similarity of two amino acids may dictate whether a certain substitution is conservative or non-conservative. Methods of determining the conservativeness of an amino acid substitution are generally known in the art and may involve substitution matrices. Commonly used substitution matrices include BLOSUM45, BLOSUM62, BLOSUM80, PAM100, PAM120, PAM160, PAM200, PAM250, but other substitution matrices or approaches may be used as considered appropriate by the skilled person. A certain substitution matrix may be preferential over the others when considering aspects such as stringency, conservation and/or divergence of related sequences (e.g. within the same species or broader), and length of the sequences in question. As used herein, a peptide sequence having a certain % similarity to another sequence will have up to that % of amino acids that are either identical or an acceptable substitution as governed by the method of similarity determination used. In some embodiments, a sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence similarity to any of the sequences disclosed herein may be used. In some embodiments, any sequence having at least 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 similar substitutions relative to any of the sequences disclosed herein may be used. As applied to antibody sequences, these similar substitutions may apply to antigen-binding regions (i.e. CDRs) or regions that do not bind to antigens or are only secondary to antigen binding (i.e. framework regions).

The term “consensus sequence” as used herein with regard to sequences refers to the generalized sequence representing all of the different combinations of permissible amino acids at each location of a group of sequences. A consensus sequence may provide insight into the conserved regions of related sequences where the unit (e.g. amino acid or nucleotide) is the same in most or all of the sequences, and regions that exhibit divergence between sequences. In the case of antibodies, the consensus sequence of a CDR may indicate amino acids that are important or dispensable for antigen binding. It is envisioned that consensus sequences may be prepared with any of the sequences provided herein, and the resultant various sequences derived from the consensus sequence can be validated to have similar effects as the template sequences.

As used herein, the term “antibody” denotes the meaning ascribed to it by one of skill in the art, and further it is intended to include any polypeptide chain-containing molecular structure with a specific shape that fits to and recognizes an epitope, where one or more non-covalent binding interactions stabilize the complex between the molecular structure and the epitope.

The term “antibody library” refers to a collection of antibodies and/or antibody fragments displayed for screening and/or combination into full antibodies. The antibodies and/or antibody fragments may be displayed on a ribosome; on a phage; or on a cell surface, in particular a yeast cell surface.

The term “compete,” as used herein with regard to an antibody or binding polypeptide, means that a first antibody or binding polypeptide, or an antigen-binding portion thereof, binds to an epitope in a manner sufficiently similar to the binding of a second antibody or binding polypeptide, or an antigen-binding portion thereof, such that the result of binding of the first antibody or binding polypeptide with its cognate epitope is detectably decreased in the presence of the second antibody or binding polypeptide compared to the binding of the first antibody or binding polypeptide in the absence of the second antibody or binding polypeptide. The alternative, where the binding of the second antibody or binding polypeptide to its epitope is also detectably decreased in the presence of the first antibody or binding polypeptide, can, but need not be the case. Regardless of the mechanism by which such competition occurs (e.g., steric hindrance, conformational change, or binding to a common epitope, or portion thereof), the skilled artisan would appreciate, based upon the teachings provided herein, that such competing antibodies or binding polypeptides are encompassed and can be useful for the methods disclosed herein.

An antibody or binding polypeptide that “preferentially binds” or “specifically binds” (used interchangeably herein) to an epitope is a term well understood in the art, and methods to determine such specific or preferential binding are also well known in the art. A molecule is said to exhibit “specific binding” or “preferential binding” if it reacts or associates more frequently, and/or more rapidly, and/or with greater duration and/or with greater affinity with a particular cell or substance than it does with alternative cells or substances. An antibody or binding polypeptide “specifically binds” or “preferentially binds” to a target if it binds with greater affinity, and/or avidity, and/or more readily, and/or with greater duration than it binds to other substances.

The term “humanized” as applies to a non-human (e.g. rodent or primate) antibodies are hybrid immunoglobulins, immunoglobulin chains or fragments thereof which contain minimal sequence derived from non-human immunoglobulin.

The term “single domain binding polypeptide” or “single domain antibody” (sdAb) as used herein refers to a single peptide strand (e.g. not bound to another peptide strand with disulfide bonds) comprising an intact immunoglobulin domain or other protein fold which can recognize antigens. Single domain binding polypeptides or sdAbs may be derived from typical heavy or light immunoglobulin chains, such as from human, or from alternative sources such as dromedaries (e.g. V_{H}H) and cartilaginous fish (e.g. V_{NAR}). In some embodiments, the single domain binding polypeptide or sdAb comprises one, two, or three complementarity determining regions (CDRs). In some embodiments, the single domain binding polypeptide or sdAb comprises one, two, or three of a CDR1, CDR2, and CDR3.

The term “single-chain variable fragment” (scFv) as used herein is a fusion protein comprising the variable regions of the heavy (VH) and light chains (VL) of an immunoglobulin, in which the VH and VL are covalently linked to form a VH:VL heterodimer. The VH and VL are either joined directly or joined by a peptide-encoding linker, which connects the N-terminus of the VH with the C-terminus of the VL, or the C-terminus of the VH with the N-terminus of the VL. The linker is usually rich in glycine for flexibility, as well as serine or threonine for solubility. Despite removal of the constant regions and the introduction of a linker, scFv proteins retain the specificity of the original immunoglobulin. Single chain Fv polypeptide antibodies can be expressed from a nucleic acid including VH- and VL-encoding sequences. In some embodiments, the VH and VL of the scFv each comprises one, two, or three CDRs. In some embodiments, the VH and VL of the scFv each comprises one, two, or three of a CDR1, CDR2, and CDR3.

In certain embodiments, definitive delineation of a CDR and identification of residues comprising the binding site of an antibody or binding polypeptide is accomplished by solving the structure of the antibody or binding polypeptide and/or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. In certain embodiments, various methods of analysis can be employed to identify or approximate the CDR regions. Examples of such methods include, but are not limited to, the Kabat definition, the Chothia definition, the IMGT approach (Lefranc et al., 2003) Dev Comp Immunol. 27:55-77), computational programs such as Paratome (Kunik et al., 2012, Nucl Acids Res. W521-4), the AbM definition, and the conformational definition.

The Kabat definition is a standard for numbering the residues in an antibody and is typically used to identify CDR regions. See, e.g., Johnson & Wu, 2000, Nucleic Acids Res., 28:214-8. The Chothia definition is similar to the Kabat definition, but the Chothia definition takes into account positions of certain structural loop regions. See, e.g., Chothia et al., 1986, J. Mol. Biol., 196:901-17; Chothia et al., 1989, Nature, 342:877-83. The AbM definition uses an integrated suite of computer programs produced by Oxford Molecular Group that model antibody structure. See, e.g., Martin et al., 1989, Proc Natl Acad Sci (USA), 86:9268-9272; “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., 1999, “Ab Initio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:732-45. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, IMGT, Paratome, AbM, and/or conformational definitions, or a combination of any of the foregoing.

The term “chimeric antigen receptor (CAR)” as used herein refers to engineered biological receptors that confers an artificial specificity in an immune cell towards a certain antigen, such as a tumor-associated antigen. An exemplary immune cell in which CARs can be used are T cells, but it is envisioned that CARs can be engineered into any amenable cytotoxic immune cell, including but not limited to T cells, Natural Killer (NK) cells, Natural Killer T (NKT) cells, dendritic cells, or macrophages. In this aspect, any disclosure pertaining to CAR T cells can also be applied to other immune cells comprising CARs. At their core, CARs comprise an extracellular antigen-recognizing domain (e.g. tumor receptor ligand, or antibody), hinge, transmembrane, and intracellular signaling domain (endodomain). Different combinations of these CAR components may result in different specificities and efficacy against certain cancer antigens.

As used herein, the terms “treating” or “treatment” (and as well understood in the art) means an approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delaying or slowing of disease progression, amelioration or palliation of the disease state, diminishment of the reoccurrence of disease, and remission, whether partial or total and whether detectable or undetectable. “Treating” and “treatment” as used herein also include prophylactic treatment. Treatment methods comprise administering to a subject a therapeutically effective amount of an active agent. The administering step may consist of a single administration or may comprise a series of administrations. The compositions are administered to the subject in an amount and for a duration sufficient to treat the subject. The length of the treatment period depends on a variety of factors, such as the severity of the condition, the age and genetic profile of the subject, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required.

The terms “effective amount” or “effective dose” as used herein refers to that amount of a recited composition or compound that results in an observable designated effect. Actual dosage levels of active ingredients in an active composition of the presently disclosed subject matter can be varied so as to administer an amount of the active composition or compound that is effective to achieve the designated response for a particular subject and/or application. The selected dosage level can vary based upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

The term “administering” includes oral administration, topical contact, administration as a suppository, parenteral, intravenous, intraperitoneal, intramuscular, intralesional, intrathecal, intranasal, subdermal, or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a first compound described herein is administered at the same time, just prior to, or just after the administration of a second compound described herein.

As used herein, the term “therapeutic target” refers to a gene or gene product that, upon modulation of its activity (e.g., by modulation of expression, biological activity, and the like), can provide for modulation of the disease phenotype. As used throughout, “modulation” is meant to refer to an increase or a decrease in the indicated phenomenon (e.g., modulation of a biological activity refers to an increase in a biological activity or a decrease in a biological activity).

As used herein, the term “standard of care”, “best practice” and “standard therapy” refers to the treatment that is accepted by medical practitioners to be an appropriate, proper, effective, and/or widely used treatment for a certain disease. The standard of care of a certain disease depends on many different factors, including the biological effect of treatment, region or location within the body, patient status (e.g. age, weight, gender, hereditary risks, other disabilities, secondary conditions), toxicity, metabolism, bioaccumulation, therapeutic index, dosage, and other factors known in the art. Determining a standard of care for a disease is also dependent on establishing safety and efficacy in clinical trials as standardized by regulatory bodies such as the US Food and Drug Administration, International Council for Harmonisation, Health Canada, European Medicines Agency, Therapeutics Goods Administration, Central Drugs Standard Control Organization, National Medical Products Administration, Pharmaceuticals and Medical Devices Agency, Ministry of Food and Drug Safety, and the World Health Organization. The standard of care for a disease may include but is not limited to surgery, radiation, chemotherapy, targeted therapy, or immunotherapy.

The term “% w/w” or “% wt/wt” means a percentage expressed in terms of the weight of the ingredient or agent over the total weight of the composition multiplied by 100.

### Antigen Binding Polypeptides

Unless otherwise specified, the complementarity determining regions (CDRs) disclosed herein follow the IMGT definition. However, the CDRs, either separately or within the context of the variable domains, can also be interpreted by Kabat, Chothia, or other definitions as understood by those of skill in the art.

Disclosed herein are mesothelin (MSLN) binding polypeptides. In some embodiments, the MSLN binding polypeptides comprise an immunoglobulin heavy chain variable domain comprising a CDR-H1, CDR-H2, and CDR-H3. In some embodiments, the CDR-H1 comprises a sequence having at least 90%, 95%, 99%, or 100% sequence identity to a sequence selected from SEQ ID NOs: 1-41. In some embodiments, CDR-H2 comprises a sequence having at least 90%, 95%, 99%, or 100% sequence identity to a sequence selected from SEQ ID NOs: 42-82. In some embodiments, the CDR-H3 comprises a sequence having at least 90%, 95%, 99%, or 100% sequence identity to a sequence selected from SEQ ID NOs: 83-123. In some embodiments, the CDR-H1 comprises a sequence having at least 90%, 95%, 99%, or 100% sequence identity to a sequence selected from SEQ ID NOs: 1-41; the CDR-H2 comprises a sequence having at least 90%, 95%, 99%, or 100% sequence identity to a sequence selected from SEQ ID NOs: 42-82; and the CDR-H3 comprises a sequence having at least 90%, 95%, 99%, or 100% sequence identity to a sequence selected from SEQ ID NOs: 83-123. In some embodiments of the MSLN binding polypeptides: 1) the CDR-H1 comprises the sequence of SEQ ID NO: 1, the CDR-H2 comprises the sequence of SEQ ID NO: 42, and the CDR-H3 comprises the sequence of SEQ ID NO: 83; 2) the CDR-H1 comprises the sequence of SEQ ID NO: 2, the CDR-H2 comprises the sequence of SEQ ID NO: 43, and the CDR-H3 comprises the sequence of SEQ ID NO: 84; 3) the CDR-H1 comprises the sequence of SEQ ID NO: 3, the CDR-H2 comprises the sequence of SEQ ID NO: 44, and the CDR-H3 comprises the sequence of SEQ ID NO: 85; 4) the CDR-H1 comprises the sequence of SEQ ID NO: 4, the CDR-H2 comprises the sequence of SEQ ID NO: 45, and the CDR-H3 comprises the sequence of SEQ ID NO: 86; 5) the CDR-H1 comprises the sequence of SEQ ID NO: 5, the CDR-H2 comprises the sequence of SEQ ID NO: 46, and the CDR-H3 comprises the sequence of SEQ ID NO: 87; 6) the CDR-H1 comprises the sequence of SEQ ID NO: 6, the CDR-H2 comprises the sequence of SEQ ID NO: 47, and the CDR-H3 comprises the sequence of SEQ ID NO: 88; 7) the CDR-H1 comprises the sequence of SEQ ID NO: 7, the CDR-H2 comprises the sequence of SEQ ID NO: 48, and the CDR-H3 comprises the sequence of SEQ ID NO: 89; 8) the CDR-H1 comprises the sequence of SEQ ID NO: 8, the CDR-H2 comprises the sequence of SEQ ID NO: 49, and the CDR-H3 comprises the sequence of SEQ ID NO: 90; 9) the CDR-H1 comprises the sequence of SEQ ID NO: 9, the CDR-H2 comprises the sequence of SEQ ID NO: 50, and the CDR-H3 comprises the sequence of SEQ ID NO: 91; 10) the CDR-H1 comprises the sequence of SEQ ID NO: 10, the CDR-H2 comprises the sequence of SEQ ID NO: 51, and the CDR-H3 comprises the sequence of SEQ ID NO: 92; 11) the CDR-H1 comprises the sequence of SEQ ID NO: 11, the CDR-H2 comprises the sequence of SEQ ID NO: 52, and the CDR-H3 comprises the sequence of SEQ ID NO: 93; 12) the CDR-H1 comprises the sequence of SEQ ID NO: 12, the CDR-H2 comprises the sequence of SEQ ID NO: 53, and the CDR-H3 comprises the sequence of SEQ ID NO: 94; 13) the CDR-H1 comprises the sequence of SEQ ID NO: 13, the CDR-H2 comprises the sequence of SEQ ID NO: 54, and the CDR-H3 comprises the sequence of SEQ ID NO: 95; 14) the CDR-H1 comprises the sequence of SEQ ID NO: 14, the CDR-H2 comprises the sequence of SEQ ID NO: 55, and the CDR-H3 comprises the sequence of SEQ ID NO: 96; 15) the CDR-H1 comprises the sequence of SEQ ID NO: 15, the CDR-H2 comprises the sequence of SEQ ID NO: 56, and the CDR-H3 comprises the sequence of SEQ ID NO: 97; 16) the CDR-H1 comprises the sequence of SEQ ID NO: 16, the CDR-H2 comprises the sequence of SEQ ID NO: 57, and the CDR-H3 comprises the sequence of SEQ ID NO: 98; 17) the CDR-H1 comprises the sequence of SEQ ID NO: 17, the CDR-H2 comprises the sequence of SEQ ID NO: 58, and the CDR-H3 comprises the sequence of SEQ ID NO: 99; 18) the CDR-H1 comprises the sequence of SEQ ID NO: 18, the CDR-H2 comprises the sequence of SEQ ID NO: 59, and the CDR-H3 comprises the sequence of SEQ ID NO: 100; 19) the CDR-H1 comprises the sequence of SEQ ID NO: 19, the CDR-H2 comprises the sequence of SEQ ID NO: 60, and the CDR-H3 comprises the sequence of SEQ ID NO: 101; 20) the CDR-H1 comprises the sequence of SEQ ID NO: 20, the CDR-H2 comprises the sequence of SEQ ID NO: 61, and the CDR-H3 comprises the sequence of SEQ ID NO: 102; 21) the CDR-H1 comprises the sequence of SEQ ID NO: 21, the CDR-H2 comprises the sequence of SEQ ID NO: 62, and the CDR-H3 comprises the sequence of SEQ ID NO: 103; 22) the CDR-H1 comprises the sequence of SEQ ID NO: 22, the CDR-H2 comprises the sequence of SEQ ID NO: 63, and the CDR-H3 comprises the sequence of SEQ ID NO: 104; 23) the CDR-H1 comprises the sequence of SEQ ID NO: 23, the CDR-H2 comprises the sequence of SEQ ID NO: 64, and the CDR-H3 comprises the sequence of SEQ ID NO: 105; 24) the CDR-H1 comprises the sequence of SEQ ID NO: 24, the CDR-H2 comprises the sequence of SEQ ID NO: 65, and the CDR-H3 comprises the sequence of SEQ ID NO: 106; 25) the CDR-H1 comprises the sequence of SEQ ID NO: 25, the CDR-H2 comprises the sequence of SEQ ID NO: 66, and the CDR-H3 comprises the sequence of SEQ ID NO: 107; 26) the CDR-H1 comprises the sequence of SEQ ID NO: 26, the CDR-H2 comprises the sequence of SEQ ID NO: 67, and the CDR-H3 comprises the sequence of SEQ ID NO: 108; 27) the CDR-H1 comprises the sequence of SEQ ID NO: 27, the CDR-H2 comprises the sequence of SEQ ID NO: 68, and the CDR-H3 comprises the sequence of SEQ ID NO: 109; 28) the CDR-H1 comprises the sequence of SEQ ID NO: 28, the CDR-H2 comprises the sequence of SEQ ID NO: 69, and the CDR-H3 comprises the sequence of SEQ ID NO: 110; 29) the CDR-H1 comprises the sequence of SEQ ID NO: 29, the CDR-H2 comprises the sequence of SEQ ID NO: 70, and the CDR-H3 comprises the sequence of SEQ ID NO: 111; 30) the CDR-H1 comprises the sequence of SEQ ID NO: 30, the CDR-H2 comprises the sequence of SEQ ID NO: 71, and the CDR-H3 comprises the sequence of SEQ ID NO: 112; 31) the CDR-H1 comprises the sequence of SEQ ID NO: 31, the CDR-H2 comprises the sequence of SEQ ID NO: 72, and the CDR-H3 comprises the sequence of SEQ ID NO: 113; 32) the CDR-H1 comprises the sequence of SEQ ID NO: 32, the CDR-H2 comprises the sequence of SEQ ID NO: 73, and the CDR-H3 comprises the sequence of SEQ ID NO: 114; 33) the CDR-H1 comprises the sequence of SEQ ID NO: 33, the CDR-H2 comprises the sequence of SEQ ID NO: 74, and the CDR-H3 comprises the sequence of SEQ ID NO: 115; 34) the CDR-H1 comprises the sequence of SEQ ID NO: 34, the CDR-H2 comprises the sequence of SEQ ID NO: 75, and the CDR-H3 comprises the sequence of SEQ ID NO: 116; 35) the CDR-H1 comprises the sequence of SEQ ID NO: 35, the CDR-H2 comprises the sequence of SEQ ID NO: 76, and the CDR-H3 comprises the sequence of SEQ ID NO: 117; 36) the CDR-H1 comprises the sequence of SEQ ID NO: 36, the CDR-H2 comprises the sequence of SEQ ID NO: 77, and the CDR-H3 comprises the sequence of SEQ ID NO: 118; 37) the CDR-H1 comprises the sequence of SEQ ID NO: 37, the CDR-H2 comprises the sequence of SEQ ID NO: 78, and the CDR-H3 comprises the sequence of SEQ ID NO: 119; 38) the CDR-H1 comprises the sequence of SEQ ID NO: 38, the CDR-H2 comprises the sequence of SEQ ID NO: 79, and the CDR-H3 comprises the sequence of SEQ ID NO: 120; 39) the CDR-H1 comprises the sequence of SEQ ID NO: 39, the CDR-H2 comprises the sequence of SEQ ID NO: 80, and the CDR-H3 comprises the sequence of SEQ ID NO: 121; 40) the CDR-H1 comprises the sequence of SEQ ID NO: 40, the CDR-H2 comprises the sequence of SEQ ID NO: 81, and the CDR-H3 comprises the sequence of SEQ ID NO: 122; or 41) the CDR-H1 comprises the sequence of SEQ ID NO: 41, the CDR-H2 comprises the sequence of SEQ ID NO: 82, and the CDR-H3 comprises the sequence of SEQ ID NO: 123.

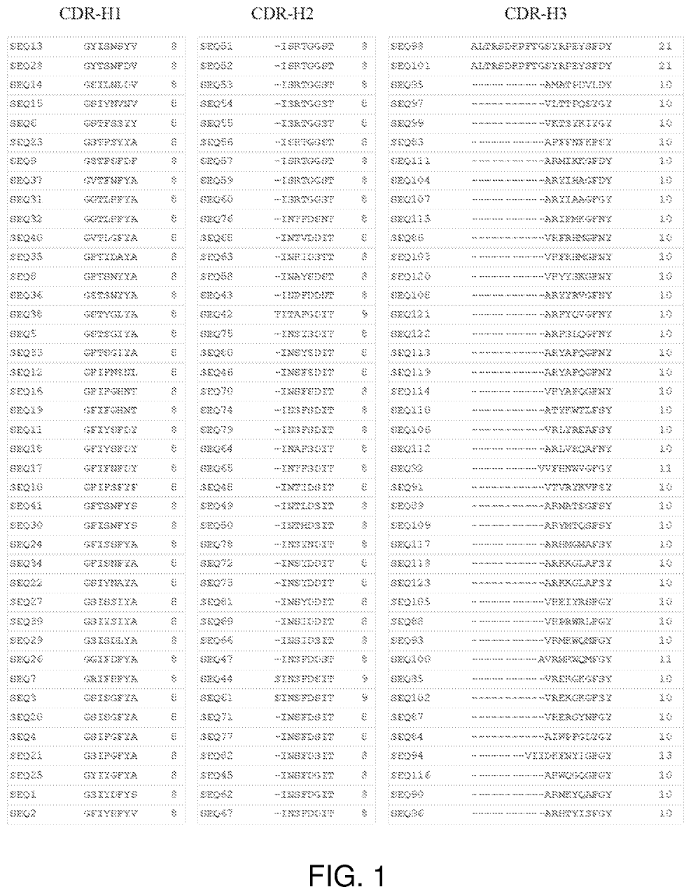

In some embodiments, the MSLN binding polypeptide comprise an immunoglobulin heavy chain variable domain comprising a CDR-H1, CDR-H2, and CDR-H3, where one or more of these CDRs are defined by a consensus sequence. The consensus sequences provided herein have been derived from the alignments of CDRs depicted in FIG. 1. However, it is envisioned that alternative alignments may be done (e.g. using global or local alignment, or with different algorithms, such as Hidden Markov Models, seeded guide trees, Needleman-Wunsch algorithm, or Smith-Waterman algorithm, or other known methods) and as such, alternative consensus sequences can be derived (including those done with a subset of the sequences provided herein).

In some embodiments, the CDR-H1 is defined by the formula X₁X₂X₃X₄X₅X₆X₇X₈, where X₁ is G; X₂ is F, G, R, S, V, or Y; X₃ is I or T; X₄ is F, L, S, or Y; X₅ is D, G, N, R, or S; X₆ is A, F, H, I, L, S, V, or Y; X₇ is D, N, or Y; X₈ is A, F, L, S, T, V, or Y. In some embodiments, the CDR-H1 comprises a sequence having at least 90%, 91%, 92%, 93%, 94%, 95%, 96%, 97%, 98%, 99%, or 100% identity to this consensus sequence. In some embodiments, the CDR-H1 comprises a sequence having 0, 1, 2, 3, 4, 5, or 6 substitutions from this consensus sequence.

In some embodiments, the CDR-H2 is defined by the formula X₁X₂X₃X₄X₅X₆X₇X₈X₉, where X₁ is no amino acid, S, or T; X₂ is I; X₃ is N, S, or, T; X₄ is A, P, R, S, or T; X₅ is F, H, I, L, T, V, or Y; X₆ is D, G, N, or S; X₇ is D, G, or S; X₈ is I, N, S, or T; X₉ is T. In some embodiments, the CDR-H2 comprises a sequence having at least 90%, 91%, 92%, 93%, 94%, 95%, 96%, 97%, 98%, 99%, or 100% identity to this consensus sequence. In some embodiments, the CDR-H2 comprises a sequence having 0, 1, 2, 3, 4, 5, or 6 substitutions from this consensus sequence.

In some embodiments, the CDR-H3 is defined by the formula X₁X₂X₃X₄X₅X₆X₇X₈X₉X₁₀X₁₁X₁₂X₁₃X₁₄X₁₅X₁₆X₁₇X₁₈X₁₉X₂₀X₂₁, where X₁ is no amino acid or A; X₂ is no amino acid or L; X₃ is no amino acid or T; X₄ is no amino acid or R; X₅ is no amino acid or S; X₆ is no amino acid or D; X₇ is no amino acid or R; X₈ is no amino acid or P; X₉ is no amino acid, F, or V; X₁₀ is no amino acid, I, or T; X₁₁ is no amino acid, A, G, I, or V; X₁₂ is A, D, S, or V; X₁₃ is F, I, K, L, M, R, T, or Y; X₁₄ is A, E, F, H, I, K, L, M, N, P, R, T, V, W, or Y; X₁₅ is A, F, I, K, M, N, P, Q, R, S, T, V, or Y; X₁₆ is A, E, F, G, H, K, L, M, N, P, Q, R, S, T, W, or Y; X₁₇ is A, D, E, F, G, I, K, L, M, Q, R, S, T, V, or Y; X₁₈ is A, D, G, I, K, L, M, N, S, or V; X₁₉ is F, L, or Y; X₂₀ is D, G, N, or S; X₂₁ is Y. In some embodiments, the CDR-H3 comprises a sequence having at least 90%, 91%, 92%, 93%, 94%, 95%, 96%, 97%, 98%, 99%, or 100% identity to this consensus sequence. In some embodiments, the CDR-H3 comprises a sequence having 0, 1, 2, 3, 4, 5, or 6 substitutions from this consensus sequence.

In some embodiments of the MSLN binding polypeptides, the heavy chain variable domain comprises an amino acid sequence having at least 90%, 95%, 99%, or 100% sequence identity to any sequence selected from SEQ ID NOs: 124-164.

In some embodiments, the MSLN binding polypeptide is humanized. In some embodiments, the MSLN binding polypeptide is a single domain antibody (sdAb).

In some embodiments, the MSLN binding polypeptide binds to MSLN with a dissociation constant (KD) of less than 1 nM, 2 nM, 5 nM, 10 nM, 15 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, or 1000 nM, or any KD within a range defined by any two of the aforementioned KD.

The binding polypeptides disclosed herein may be obtained from an antibody library. In some embodiments, the antibody library is an immune antibody library, a naïve antibody library, a synthetic antibody library, or a semi-synthetic antibody library. In some embodiments, the antibody library comprises antibodies derived from human, or antibodies that are not immunogenic in humans, or both. In some embodiments, the antibody library comprises antibodies that are humanized, e.g. from mouse, rat, guinea pig, rabbit, cat, dog, cow, horse, sheep, goat, horse, donkey. In some embodiments, the antibody library comprises single domain antibodies (sdAb), nanobodies, V_{H}H fragments, V_{NAR} fragments, single-chain variable fragments (scFv), camelid antibodies, or cartilaginous fish antibodies, or any combination thereof. One exemplary library that can be used is a fully humanized, synthetic, sdAb library, but any other antibody library that can be prepared or is available can be used for the methods disclosed herein. In some embodiments, the antibody library comprises sdAb. In some embodiments, the antibody library comprises at least 100, 500, 1000, 2000, 3000, 4000, 5000, 6000, 7000, 8000, 9000, 10000, 20000, 30000, 40000, 50000, 60000, 70000, 80000, 90000, 100000, 500000, or 1000000 unique antibodies, or any number of antibodies within a range defined by any two of the aforementioned number of antibodies.

Antibody libraries may be generated computationally or using machine learning processes. An exemplary method of generating an antibody library computationally includes modifying a universal V_{H}H framework with synthetic diversity in one or more complementary determining regions (CDRs), such as CDR1, CDR2, or CDR3, or any combination thereof. The diversity of the CDRs are introduced by randomizing the library of sequences encoding for the antibodies with degenerate codons. For example, an NNK codon library can be employed, where the NNK codon comprises N (25% mix of A/T/C/G) and K (50% mix of T/G). In some embodiments, the NNK codon library is constructed with all possible amino acids, or with some amino acids (e.g. cysteine) and stop codon combinations excluded. Other degenerate codon mixes can be substituted for said NNK codon library with minimal experimentation. In other embodiments, the antibody library can be generated using a trimer codon mix, which improves balanced representation of sense codons while reducing the chance of stop codons, improving efficiency of antibody generation and testing. In some embodiments, artificial intelligence-based prediction can be used to randomize specific binding pockets of the antibodies using available binding models or structure data.

In some embodiments, panning the antibody library comprises screening for the candidate binding polypeptides by phage display, yeast display, bacterial display, ribosome display, or mRNA display, or any combination thereof. In some embodiments, panning the antibody library comprises one or more rounds of selection, wherein the candidate binding polypeptides are selected for specificity towards a cancer-associated antigen (e.g. MSLN) or cells or tissues displaying the cancer-associated antigen. In some embodiments, the candidate binding polypeptides are selected under conditions including but not limited to tumor microenvironment-like conditions, immunosuppressive conditions, low or high pH, low or high oxygen concentrations, low or high temperatures, low or high viscosity, or any combination thereof, or for specificity towards modified or derivative forms of the one or more cancer-associated antigens. In some embodiments, the immunosuppressive conditions may comprise the presence of tumor-associated macrophages (TAMs), myeloid-derived suppressor cells (MDSCs), tumor-associated neutrophils (TANs), cancer-associated fibroblasts (CAFs), or other immunosuppressive cells, or the presence of adenosine, or both.

In some embodiments, the chimeric antigen receptor cells are from a cell line (e.g. Jurkat). In some embodiments, the chimeric antigen receptor cells are derived from a subject. In some embodiments, the subject has a cancer. In some embodiments, the subject has a cancer, and that cancer expresses any one or more of the cancer-associated antigens disclosed herein (e.g., MSLN). In some embodiments, the cancer is breast cancer, colorectal cancer, kidney cancer, liver cancer, lung cancer, brain cancer, pancreatic cancer, bladder cancer, testicular cancer, prostate cancer, gastric cancer, ovarian cancer, head and neck cancer, gallbladder cancer, a hematologic malignancy, or any combination thereof. In some embodiments, the hematologic malignancy may comprise leukemia, acute lymphoblastic leukemia, acute myelogenous leukemia, chronic myelogenous leukemia, chronic lymphocytic leukemia, hairy cell leukemia, lymphoma, Hodgkin's disease, Non-Hodgkin lymphoma, or multiple myeloma. In some embodiments, the subject is a mammal, such as a human, cat, dog, mouse, rat, hamster, rodent, cow, pig, horse, goat, sheep, donkey, or monkey. In some embodiments, the subject is a human.

### Chimeric Antigen Receptors (CARs) and Cells

Also disclosed herein are chimeric antigen receptors (CARs) comprising any one or more of the MSLN binding polypeptides disclosed herein.

In some embodiments, the CAR comprises at least two binding polypeptides and the CAR is a multivalent CAR. In some embodiments, the CAR comprises two binding polypeptides and the CAR is a bivalent CAR. In some embodiments, the CAR comprises three binding polypeptides and the CAR is a trivalent CAR.

In some embodiments, the CAR further comprises one or more signal peptides, linkers with various lengths and composition, hinges, transmembrane domains, costimulatory domains, signaling domains, cytoplasmic domains, functionality signals, proliferation signals, anti-exhaustion signals, anti-inhibitory receptors, tumor/cancer homing proteins, or regulatory molecules, or any combination thereof. In some embodiments, the hinges comprise CD3ζ, CD4, CD8 or CD28 hinges, or computationally designed synthetic hinges with various lengths. In some embodiments, the transmembrane domains comprise CD3ζ, CD4, CD8 or CD28 transmembrane domains, or computationally designed synthetic transmembrane domains. In some embodiments, the costimulatory domains comprise CD8, CD28, ICOS, 4-1BB, OX40 (CD134), CD27, CD40, CD40L, TLR or other TNFR superfamily member or Ig superfamily member costimulatory domains, or other signaling via cytoplasmic domains of IL-2Rβ, IL-15R-α, MyD88, or CD40 or any other Toll-like receptor or IL-1 receptor signaling pathway members.

In some embodiments, the CARs disclosed herein are constructed by assembling CAR expression constructs from nucleic acids encoding for any one of the binding polypeptides disclosed herein and a mixture of compatible nucleic acids encoding for different CAR modules. In some embodiments, different combinations of CARs are produced for use in a CAR library for screening for CAR efficacy (in vitro or in vivo). In some embodiments, unique CARs are produced separately. In some embodiments, the CARs are specific for one target. In some embodiments, the CARs are specific for at least 1, 2, 3, 4, 5, 6, 7, 8, 9, or 10 targets. In some embodiments, the CARs are bi-specific or tri-specific.

To construct any one of CARs disclosed herein, the nucleic acids encoding for the binding polypeptides identified by panning of the antibody library are assembled into CAR expression constructs with other CAR modules. In some embodiments, the CAR expression constructs are assembled using multi-fragment assembly reactions known in the art. One exemplary method of assembling CAR expression constructs involves using Type IIS restriction enzymes to generate nucleic acid fragments with compatible overhang sequences and ligating the nucleic acid fragments with a ligase. As Type IIS restriction enzymes cleave outside of their recognition sites, multiple compatible nucleic acid fragments may be prepared simultaneously. In other embodiments, the CAR expression constructs can be assembled by overlap extension PCR. It is envisioned that any other method of assembling nucleic acid constructs from more than one nucleic acid fragment can be employed. In some embodiments, the different CAR modules comprise signal peptides, linkers, hinges, transmembrane domains, costimulatory domains, activation domains, signaling domains, cytoplasmic domains, functionality signals, proliferation signals, anti-exhaustion signals, anti-inhibitor receptors, cancer homing proteins, or regulatory molecules, or any combination thereof. Some exemplary hinges comprise CD8 hinge, CD28 hinge, IgG1 hinge, or IgG4 hinge. Some exemplary transmembrane domains comprise CD3ζ transmembrane domain, CD8α transmembrane domain, CD4 transmembrane domain, CD28 transmembrane domain, or ICOS transmembrane domain. Some exemplary costimulatory domains comprise CD8 costimulatory domain, CD28 costimulatory domain, 4-1BB costimulatory domain, OX40 (CD134) costimulatory domain, ICOS costimulatory domain, CD27 costimulatory domain, CD40 costimulatory domain, CD40L costimulatory domain, TLR costimulatory domains, MYD88-CD40 costimulatory domain, or KIR2DS2 costimulatory domain. In some embodiments, the different CAR modules are derived from CD8, CD28, 4-1BB, CD3ζ, or any combination thereof. The CAR may also be modified with various additions, including but not limited to cytokines, chemokines, cytokine receptors, chemokine receptors, antigen receptors or ligands, antibodies, or enzymes.

Also disclosed herein are chimeric antigen receptor (CAR) cells comprising any one of the CARs disclosed herein. In some embodiments, the CAR cell is a CAR-T cell. In some embodiments, the CAR cell is derived from a subject or from a cell line. In some embodiments, the subject has a cancer. In some embodiments, the subject has a cancer, and that cancer expresses any one or more of the cancer-associated antigens disclosed herein (e.g., MSLN). In some embodiments, the cancer is breast cancer, colorectal cancer, kidney cancer, liver cancer, lung cancer, brain cancer, pancreatic cancer, bladder cancer, testicular cancer, prostate cancer, gastric cancer, ovarian cancer, head and neck cancer, gallbladder cancer, a hematologic malignancy, or any combination thereof. In some embodiments, the hematologic malignancy may comprise leukemia, acute lymphoblastic leukemia, acute myelogenous leukemia, chronic myelogenous leukemia, chronic lymphocytic leukemia, hairy cell leukemia, lymphoma, Hodgkin's disease, Non-Hodgkin lymphoma, or multiple myeloma.

### Nucleic Acids

Also disclosed herein are nucleic acids that encode for a polypeptide. In some embodiments, the polypeptide is a binding polypeptide. In some embodiments, the polypeptide is a single domain binding polypeptide. In some embodiments, the polypeptide is any one of the binding polypeptides disclosed herein. In some embodiments, the polypeptide comprises a sequence having at least 90%, 95%, 99%, or 100% sequence identity to: any one or more of the MSLN binding polypeptides disclosed herein. In some embodiments, the polypeptide is any one of the CARs disclosed herein.

Any one of the nucleic acids that encode for a binding polypeptide can be prepared by recombinant DNA technology, synthetic chemistry techniques, or a combination thereof. For example, sequences of nucleic acids encoding for the binding polypeptide may be cloned into an expression vector using standard molecular techniques known in the art. Sequences can be obtained from other vectors encoding the desired protein sequence, from PCR-generated fragments using respective template nucleic acids, or by assembly of synthetic oligonucleotides encoding the desired sequences. In some embodiments, the expression vector may be a CAR expression vector, in which it is provided to an immune cell so that it expresses the CAR. In some embodiments, the expression vector may be an expression vector suited for large scale antibody or binding polypeptide production, from which the peptide products can be isolated for further use.

Expression of binding polypeptides or CARs may be confirmed by nucleic acid or protein assays known in the art. For example, the presence of transcribed mRNA of binding polypeptides or CARs can be detected and/or quantified by conventional hybridization assays (e.g. Northern blot analysis), amplification procedures (e.g. RT-PCR), SAGE (U.S. Pat. No. 5,695,937), and array-based technologies (see e.g. U.S. Pat. Nos. 5,405,783, 5,412,087 and 5,445,934), using probes complementary to any region of a polynucleotide that encodes for the binding polypeptides or CARs. Expression of the binding polypeptides or CARs can also be determined by examining the expressed peptide. A variety of techniques are available in the art for protein analysis. They include but are not limited to radioimmunoassays, ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunofluorescent assays, and SDS-PAGE.

### Methods of Use or Treatment

Also disclosed herein are methods of treating a cancer in a subject in need thereof. In some embodiments, the methods comprise administering a chimeric antigen receptor cell to the subject. In some embodiments, the methods comprise administering any one of the chimeric antigen receptor cells disclosed herein. In some embodiments, the chimeric antigen receptor cell expresses and/or comprises any one or more of the MSLN binding polypeptides disclosed herein. In some embodiments, the chimeric antigen receptor cell is a CAR-T cell. In some embodiments, the chimeric antigen receptor cell is derived from the subject and is autologous to the subject. In some embodiments, the chimeric antigen receptor cell is allogeneic to the subject. In some embodiments, the chimeric antigen receptor cell is from a cell line (e.g. Jurkat). In some embodiments, the subject is a mammal, such as a human, cat, dog, mouse, rat, hamster, rodent, cow, pig, horse, goat, sheep, donkey, or monkey. In some embodiments, the subject is a human. In some embodiments, the subject has a cancer, and that cancer expresses any one or more of the cancer-associated antigens disclosed herein (e.g., MSLN). In some embodiments, the cancer is breast cancer, colorectal cancer, kidney cancer, liver cancer, lung cancer, brain cancer, pancreatic cancer, bladder cancer, testicular cancer, prostate cancer, gastric cancer, ovarian cancer, head and neck cancer, gallbladder cancer, a hematologic malignancy, or any combination thereof. In some embodiments, the hematologic malignancy may comprise leukemia, acute lymphoblastic leukemia, acute myelogenous leukemia, chronic myelogenous leukemia, chronic lymphocytic leukemia, hairy cell leukemia, lymphoma, Hodgkin's disease, Non-Hodgkin lymphoma, or multiple myeloma. In some embodiments, the chimeric antigen receptor cell is administered parenterally.

In some embodiments, the chimeric antigen receptor cell is administered once per day, twice per day, three times per day or more. In some embodiments, the chimeric antigen receptor cell is administered daily, every day, every alternate day, five days a week, once a week, every other week, two weeks per month, three weeks per month, once a month, twice a month, three times per month, or more. In some embodiments, the immune cell is administered for at least 1 month, 2 months, 3 months, 4 months, 5 months, 6 months, 7 months, 8 months, 9 months, 10 months, 11 months, 12 months, 18 months, 2 years, 3 years, or more.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The ranges for administration are merely suggestive, as the number of variables in regard to an individual treatment regime is large, and considerable excursions from these recommended values are not uncommon. Such dosages is altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### EXAMPLES

Some aspects of the embodiments discussed above are disclosed in further detail in the following examples, which are not in any way intended to limit the scope of the present disclosure. Those in the art will appreciate that many other embodiments also fall within the scope of the invention, as it is described herein above and in the claims.

### Example 1. Methodology

Plasmid Preparation and Quality Check (QC)

The Maxi Plasmid Purification kit (Zymo, D4203) was used for CAR plasmid preparation. Plasmid concentration and quality was analyzed by Nanodrop (260/280 ratio and 260/230 ratio) and the ToxinSensor Chromogenic LAL Endotoxin Assay (Genescript, L00350). Good-quality plasmid DNA will have an A260/A280 ratio of 1.8-2.0, an A260/A230 ratio greater than 2.0, and less than 0.1 EU/μg of endotoxin.

Plasmid Assembly and QC

All CARs were synthesized by Genewiz. Synthetic single domain antibody genes encoding antibodies which bind to mesothelin were cloned into the pLenti vector and the construct was confirmed using Sanger sequencing. The CAR constructs contain a signal peptide (e.g. CD8 signal peptide), hinge (e.g. CD8α stalk), transmembrane domain (e.g. CD8 transmembrane domain or CD28 transmembrane domain), and signaling domains (e.g. 4-1BB costimulatory domain or CD3ζ signaling domain) and the anti-MSLN sdAb.

To monitor CAR expression levels, the expressed cassette of the CAR plasmids can also be engineered to express fluorescent eGFP+T2A self-cleaving peptide sequences. Alternatively, truncated CD19 or truncated EGFR cassettes can be used to monitor by antibody detection. Human sequences (e.g. CD8, 4-1BB, CD3ζ) are accessible on GenBank.

Virus Production and Titration

To produce the lentivirus with the anti-MSLN CAR construct, human embryonic kidney 293T (HEK293T) cells were co-transfected with CAR transgene-encoding pLenti transfer plasmid and one or more necessary packaging plasmids (i.e. encoding for Gag, Pol, Rev, VSVG, and optionally Tat). The supernatants of the HEK293T cultures were collected at either 48 or 72 hours after transfection, centrifuged, and filtered with a 0.45 μm filter.

Virus titration was done in Jurkat cells transduced with diluted lentivirus collections. After 48 hours, transduced Jurkat cells were stained with biotinylated recombinant protein L and phycoerythrin (PE)-conjugated streptavidin, and anti-MSLN CAR abundance was measured by flow cytometry.

T Cell Transduction and Expansion

Human PBMCs were isolated from peripheral blood of healthy human donors by density gradient centrifugation with the Lymphoprep reagent (StemCell Technologies). PBMCs were resuspended at 1×10⁶ cells/mL in X-VIVO 15 serum-free hematopoietic medium (Lonza, 04-418QCN) with 10 ng/mL IL-2 (Novoprotein, GMP-CD66) and 10 ng/mL IL-7 (Novoprotein, GMP-CD47). PBMCs were stimulated with 50 ng/mL anti-CD3 antibody (Novoprotein, GMP-A018) for 24 hours. Then, PBMCs were transduced with anti-MSLN CAR lentivirus (at MOI of 1-10) and expanded in appropriate flasks for 9-10 days in RPMI 1640 basal medium supplemented with 10% fetal bovine serum. T cells were rested for 24 hours at 37° C. before any assay. CAR surface levels, CD3, and CD4/CD8 ratios (using antibodies from Biolegend, 317344, 301012, and 317412) were measured 12 or 14 days after initial stimulation of PBMCs with the anti-CD3 antibody.

T Cell Cryopreservation and Thawing

After 14 days of CAR-T expansion, anti-MSLN CAR-T cells were centrifuged and the supernatant was discarded. The cell pellet was resuspended in chilled CryoStor CS10 (StemCell Technologies, 07930) at a viable cell density of 5×10⁷ cells/mL. Aliquots of cell suspension were dispensed into cryovials. The cryovials were cooled with a 1° C./minute decrease. The frozen cells were transferred to liquid nitrogen.

To thaw, the cells are quickly thawed in a 37° C. water bath with gentle agitation. The thawed cells are transferred to a 50 mL conical tube and washed by adding 20 mL of fresh growth medium dropwise. The cells are centrifuged and resuspended in X-VIVO 15 medium at a cell density of 1×10⁶ cells/mL.

Cytotoxicity Assay

The cytotoxicity of anti-MSLN CAR T-cells was determined by standard luciferase-based assays. Briefly, target cells expressing firefly luciferase were co-cultured with CAR-T cells in triplicate at the indicated effector:target ratios using white-walled 96-well plates with 2×10⁴ target cells in a total volume of 100 μL per well in X-VIVO 15 medium. Target cells alone as a control were plated at the same cell density. After 48 hours of co-culture, 100 μL of luciferase substrate (ONE-Glo, Promega) was directly added to each well. Emitted light was detected with a luminescence plate reader.

In a similar luciferase based cytotoxicity assay, target cancer cells engineered to overexpress luciferase were prepared in a cell culture medium (such as RPMI) and seeded on to 96 well plates with 5×10⁴ to 5×10⁶ cells in a volume of 100 μL/well, and incubated at 37° C. for 24 hours. Then, CAR T-cells were seeded in corresponding wells with CAR T-cells at various effector:target (E:T) ratios (e.g., 5:1, 1:1, and 1:5) in 100 μL/well. The co-cultures were then incubated at 37° C. for 24 or 48 hours. Subsequently, the cells were processed with the Luciferase Assay System (Promega, E1501) according to manufacturer's instructions using a plate reader. Luminescence was read in endpoint mode using a BioTek Synergy H4 hybrid microplate reader for 10 seconds. Percentage of specific lysis was calculated using the luciferase activity of digitonin-treated cells as maximum cell death and untreated cells as spontaneous cell death, using the formula % specific lysis=100%×[(experimental luminescence−spontaneous cell death luminescence)/(maximum cell death luminescence−spontaneous cell death luminescence)].

Additional information regarding exemplary luciferase cytotoxicity assays may be found in Matta H et al. “Development and characterization of a novel luciferase based cytotoxicity assay” Scientific Reports (2018) 8,199, which is hereby expressly incorporated by reference in its entirety.

IFN-γ Evaluation

1×10⁶ per well of anti-MSLN CAR T-cells were stimulated with target cells at indicated effector:target cell ratios in 6-well tissue culture plates for 24 hours. Interferon gamma (IFN-γ) and IL-2 secretion was quantitated through enzyme-linked immunosorbent assay (ELISA) using the human IL-2 ELISA Kit II (BD, 550611) and human IFN-γ ELISA Kit II (BD, 550612)

CD25/CD69 Assay

1×10⁶ per well of anti-MSLN CAR T-cells were stimulated with target cells at indicated effector:target cell ratios in 6-well tissue culture plates for 24 hours. Cells were stained with LIVE/DEAD Fixable Near-IR Dead Cell Stain Kit (ThermoFisher) to label dead cells and with anti-CD3, and CAR-T cells were identified as CD3+GFP+cells by flow cytometry. CD69 and CD25 on CAR-T cells were stained with Alexa Fluor 700 anti-human CD69 antibody (Biolegend) and PE anti-CD25 antibody (Biolegend).

Sequencing of CAR T Cell Libraries

Cells were collected from anti-MSLN CAR-T library screens. Genomic DNA (gDNA) was extracted using the Blood/Cell Genome DNA Mini kit (Tiangen, DP304-03). 75 ng of gDNA was used for PCT to amplify the CAR region of the library. The amplified region was TA cloned with the TA/Blunt-Zero Cloning kit (Vazyme, C601) and sequenced.

### Example 2. Exemplary Anti-MSLN CAR T-Cell Lines are Effective Against Cancers

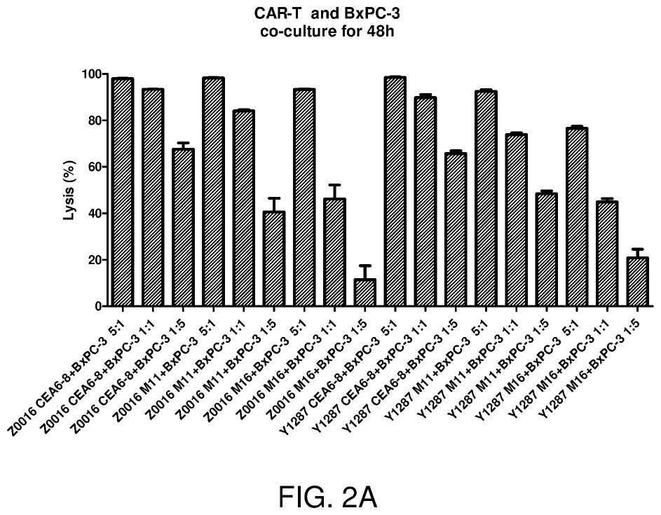

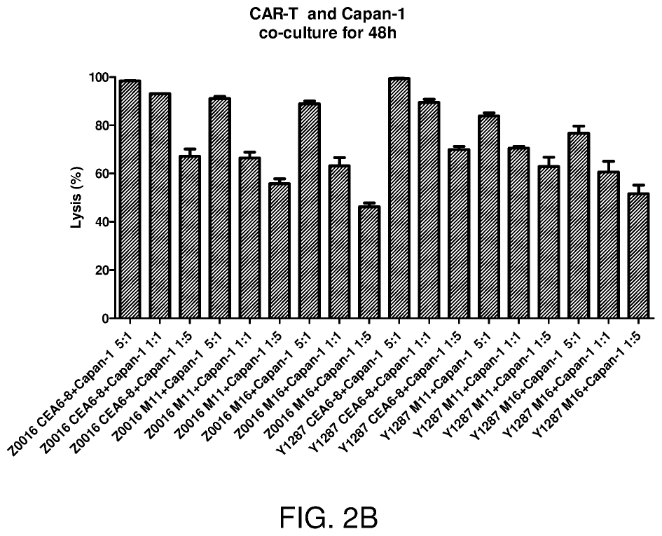

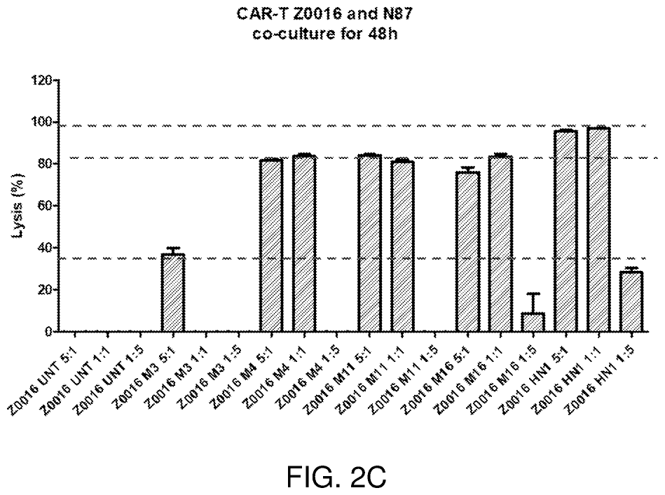

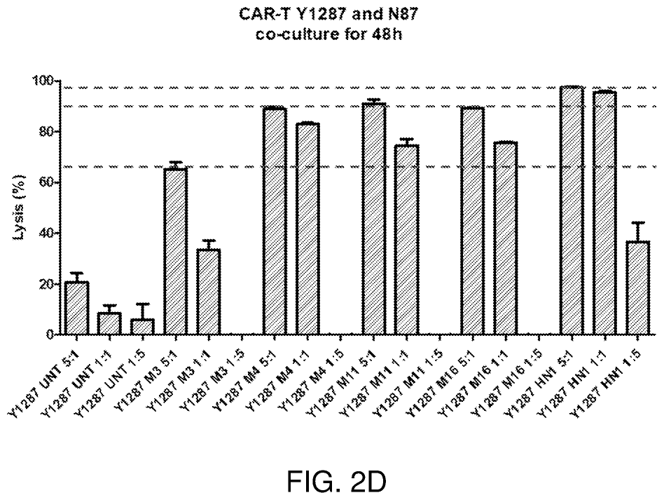

Several anti-MSLN CAR T-cell lines were prepared from the anti-MSLN binders disclosed herein. Constructs were expressed in T-cells isolated from two donors, denoted Z0016 and Y1287. Cytotoxicity assays were performed as described in Example 1, at effector:target ratios of 1:5, 1:1, and 5:1. These anti-MSLN CAR T-cell lines showed cytotoxic efficacy against BxPC-3 pancreatic cancer cells (FIG. 2A), Capan-1 pancreatic cancer cells (FIG. 2B), and NCI-N87 (N87) gastric cancer cells (FIG. 2C). Untransfected T-cells (UNT) were used as negative control.

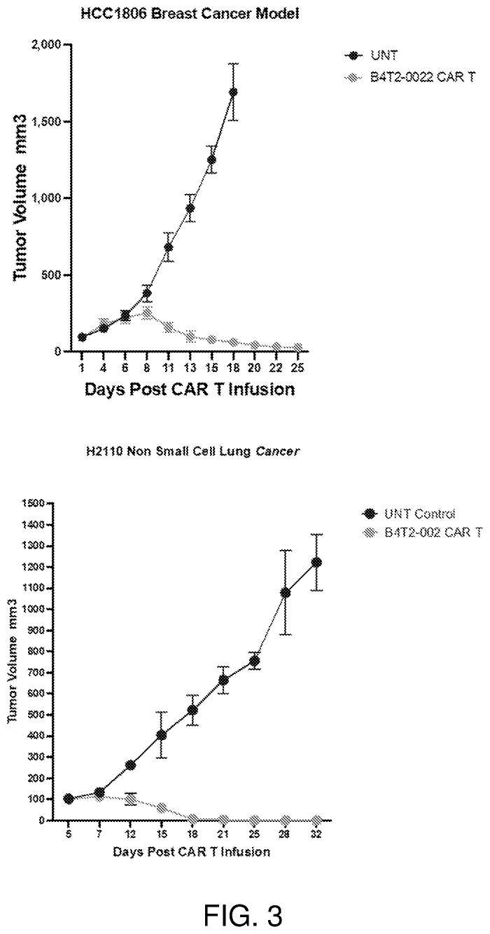

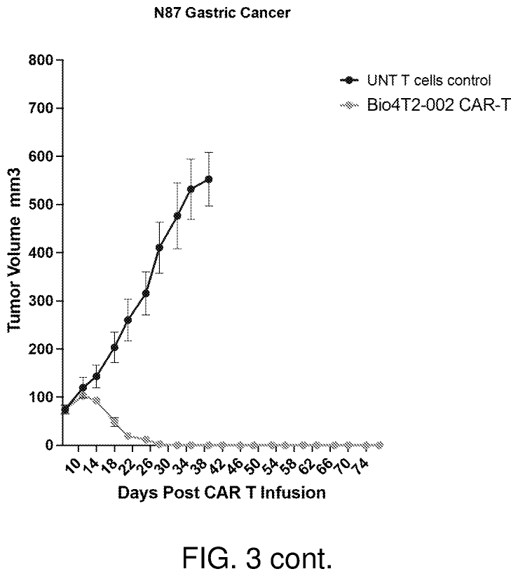

An exemplary anti-MSLN CAR T-cell line B4T2-002 was tested in in vivo NSG mouse models. In a cell line-derived xenograft model, administration of B4T2-002 resulted in dramatic clearance of the xenograft in HCC1806 breast cancer, H2110 non-small cell lung cancer, and N87 gastric cancer models, whereas tumor growth in control mice progressed (FIG. 3).

### Example 3. Anti-MSLN CAR-T Cells for the Treatment of Cancer

A patient presents with a cancer, for example, a cancer that expresses or overexpresses MSLN (e.g., such that MSLN can be used as a biomarker to detect and target the cancer). In some cases, the cancer is breast cancer, colorectal cancer, kidney cancer, liver cancer, lung cancer, brain cancer, pancreatic cancer, bladder cancer, testicular cancer, prostate cancer, gastric cancer, ovarian cancer, head and neck cancer, gallbladder cancer, a hematologic malignancy, or any combination thereof. In some cases, the hematologic malignancy may comprise leukemia, acute lymphoblastic leukemia, acute myelogenous leukemia, chronic myelogenous leukemia, chronic lymphocytic leukemia, hairy cell leukemia, lymphoma, Hodgkin's disease, Non-Hodgkin lymphoma, or multiple myeloma.

One or more of the CAR T-cells disclosed herein, which may include any one or more of the anti-MSLN CARs and/or anti-MSLN binders, are administered to the patient enterally, orally, intranasally, parenterally, intravenously, intraperitoneally, intramuscularly, intra-arterially, intraventricularly, intradermally, intralesionally, intracranially, intrathecally, or subcutaneously.

The CAR T-cells are administered as doses in the amount of 10⁴, 10⁵, 10⁶, 10⁷, 10⁸, or 10⁹ cells per dose, or any number of cells within a range defined by any two of the aforementioned cells per dose, or any number of cells that is effective and/or safe as determined by a trained medical practitioner. The doses are administered every 1, 2, 3, 4, 5, 6, 7, 8, 9, 10, 11, 12, 24, 36, or 48 days, or weeks, or any time within a range defined by any two of the aforementioned times, or any timing of dosing that is effective and/or safe as determined by a trained medical practitioner.

An improvement of the cancer or symptoms thereof is observed in the patient following administration of the one or more CAR T-cells. Administration of the CAR T-cells may be performed in conjunction with another therapy for cancer, including but not limited to immunotherapy, chemotherapy, radiation therapy, surgery, photodynamic therapy, or targeted therapy.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “ a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

Sequence List
MSLN sequences

| | | SEQ | | SEQ | | SEQ |
|---|---|---|---|---|---|---|
| | | ID | | ID | | ID |
| Name | CDR-H1 | NO | CDR-H2 | NO | CDR-H3 | NO |
| MSLN-A01 | GSIYDFYS | 1 | TITAFGDIT | 42 | AFFFNFKFSY | 83 |
| MSLN-B03 | GFIYRFYV | 2 | INPFDDNT | 43 | AIWPFGDYGY | 84 |
| MSLN-A06 | GSISGFYA | 3 | SINSFDSIT | 44 | VREKGKGFSY | 85 |
| MSLN-H04 | GSIFGFYA | 4 | INSFDGIT | 45 | VRFRHMGENY | 86 |
| MSLN-C05 | GSTSGIYA | 5 | INSFSDIT | 46 | VRERGYNFGY | 87 |
| MSLN-E05 | GSTFSSYY | 6 | INSFDDST | 47 | VRPRWRLFGY | 88 |
| MSLN-D01 | GRIFRFYA | 7 | INTIDSIT | 48 | ARNATSGESY | 89 |
| MSLN-D08 | GFTSNYYA | 8 | INTLDSIT | 49 | ARNKYQAFGY | 90 |
| MSLN-H02 | GSTFSFDF | 9 | INTHDSIT | 50 | VTVRYKVFSY | 91 |
| MSLN-A02 | GFIFSFYF | 10 | ISRTGGST | 51 | VVFHNWVGFGY | 92 |
| MSLN-H08 | GFIYSFDY | 11 | ISRTGGST | 52 | VRMRWQMFGY | 93 |
| MSLN-A03 | GFIFNSNL | 12 | ISRTGGST | 53 | VIIDKENYIGFGY | 94 |
| MSLN-A05 | GYISNSYV | 13 | ISRTGGST | 54 | AMATPDVLDY | 95 |
| MSLN-F01 | GSILNLDV | 14 | ISRTGGST | 55 | ARHTYISFGY | 96 |
| MSLN-G01 | GSIYNVNV | 15 | ISRTGGST | 56 | VLTTPQSYGY | 97 |
| MSLN-A09 | GFIFGHNT | 16 | ISRTGGST | 57 | ALTRSDRPFTGSYRPEYSEDY | 98 |
| MSLN-H06 | GFIFNFDY | 17 | INAYSDST | 58 | VKTSYKIYGY | 99 |
| H004-11- | GFIYSEDY | 18 | ISRTGGST | 59 | AVRMRWQMFGY | 100 |
| 41BB | | | | | | |
| H004-16- | GFIFGHNT | 19 | ISRTGGST | 60 | ALTRSDRPFTGSYRPEYSFDY | 101 |
| 41BB | | | | | | |
| H004-3- | GSISGFYA | 20 | SINSFDSIT | 61 | VREKGKGFSY | 102 |
| 41BB | | | | | | |
| H004-4- | GSIFGFYA | 21 | INSFDGIT | 62 | VRFRHMGFNY | 103 |
| 41BB | | | | | | |
| H004BC2-1 | GSIYNAYA | 22 | INRIDSTT | 63 | ARYIHAGFDY | 104 |
| H004BC2-2 | GSTFSYYA | 23 | INAFSDIT | 64 | VREIYRSFGY | 105 |
| H004BC2-3 | GFISSFYA | 24 | INTFSDIT | 65 | VRLYREAFSY | 106 |
| H004BC2-4 | GYIYGFYA | 25 | INSIDSIT | 66 | ARYIAAGFGY | 107 |
| H004BC2-5 | GGIFDFYA | 26 | INSFDDIT | 67 | ARYYRVGENY | 108 |
| H004BC2-6 | GSISSIYA | 27 | INTVDDIT | 68 | ARYMTQSFSY | 109 |
| H004BC2-7 | GYTSNFDV | 28 | INSIDDIT | 69 | ATYKWTLFSY | 110 |
| H004BC2-8 | GSISDLYA | 29 | INSFSDIT | 70 | ARMIKKGFDY | 111 |
| H004BC2-9 | GFISNFYS | 30 | INSFDSIT | 71 | ARLVRQAFNY | 112 |
| H004BC2-10 | GGTLRFYA | 31 | INSYDDIT | 72 | ARYAFQGENY | 113 |
| H004BC2-11 | GGTLRFYA | 32 | INSYDDIT | 73 | VRYAFQGENY | 114 |
| H004BC2-12 | GFTSGIYA | 33 | INSFSDIT | 74 | ARIFMKGENY | 115 |
| H004BC2-13 | GFISNFYA | 34 | INSYSDIT | 75 | ARWQGQGFGY | 116 |
| H004BC2-14 | GFTYDAYA | 35 | INTFDSNT | 76 | ARHMGMAFSY | 117 |
| H004BC2-15 | GSTSNYYA | 36 | INSFDSIT | 77 | ARKKGLAFSY | 118 |
| H004BC2-16 | GVTFNFYA | 37 | INSYNDIT | 78 | ARYAFQGENY | 119 |
| H004BC2-17 | GSTYGLYA | 38 | INSFSDIT | 79 | VRYYSKGFNY | 120 |
| H004BC2-18 | GSIYSIYA | 39 | INSYSDIT | 80 | ARFYQVGENY | 121 |
| H004BC2-19 | GVTLGFYA | 40 | INSYDDIT | 81 | ARFSLQGENY | 122 |
| H004BC2-20 | GFTSNFYS | 41 | INSFDSIT | 82 | ARKKGLAFSY | 123 |

| | | SEQ |
|---|---|---|
| | | ID |
| Name | Heavy Chain Variable Domain Sequence | NO: |
| MSLN-A01 | EVQLVESGGGLVQPGGSLRLSCAASGSIYDFYSMGWFRQAPGKGRELVAATITAFGD | 124 |
| | ITYYPDSVEGRFTISRDNAKRMVYLQMNSLRAEDTAVYYCAFFFNFKFSYWGQGTQV | |
| | TVSS | |
| MSLN-B03 | EVQLVESGGGLVQPGGSLRLSCAASGFIYRFYVMGWFRQAPGKGRELVAAINPEDDN | 125 |
| | TYYPDSVEGRFTISRDNAKRMVYLQMNSLRAEDTAVYYCAIWPFGDYGYWGQGTQVT | |
| | VSS | |
| MSLN-A06 | EVQLVESGGGLVQPGGSLRLSCAASGSISGFYAMGWFRQAPGKGRELVAASINSFDS | 126 |
| | ITYYPDSVEGRFTISRDNAKRMVYLQMNSLRAEDTAVYYCVREKGKGFSYWGQGTQV | |
| | TVSS | |
| MSLN-H04 | EVQLVESGGGLVQPGGSLRLSCAASGSIFGFYAMGWFRQAPGKGRELVAAINSEDGI | 127 |
| | TYYPDSVEGRETISRDNAKRMVYLQMNSLRAEDTAVYYCVRFRHMGFNYWGQGTQVT | |
| | VSS | |
| MSLN-C05 | EVQLVESGGGLVQPGGSLRLSCAASGSTSGIYAMGWFRQAPGKGRELVAAINSESDI | 128 |
| | TYYPDSVEGRFTISRDNAKRMVYLQMNSLRAEDTAVYYCVRERGYNFGYWGQGTQVT | |
| | VSS | |
| MSLN-E05 | EVQLVESGGGLVQPGGSLRLSCAASGSTFSSYYMGWFRQAPGKGRELVAAINSFDDS | 129 |
| | TYYPDSVEGRFTISRDNAKRMVYLQMNSLRAEDTAVYYCVRPRWRLFGYWGQGTQVT | |
| | VSS | |
| MSLN-D01 | EVQLVESGGGLVQPGGSLRLSCAASGRIFRFYAMGWFRQAPGKGRELVAAINTIDSI | 130 |
| | TYYPDSVEGRETISRDNAKRMVYLQMNSLRAEDTAVYYCARNATSGFSYWGQGTQVT | |
| | VSS | |
| MSLN-D08 | EVQLVESGGGLVQPGGSLRLSCAASGFTSNYYAMGWFRQAPGKGRELVAAINTLDSI | 131 |
| | TYYPDSVEGRFTISRDNAKRMVYLQMNSLRAEDTAVYYCARNKYQAFGYWGQGTQVT | |
| | VSS | |
| MSLN-H02 | EVQLVESGGGLVQPGGSLRLSCAASGSTFSFDFMGWFRQAPGKGRELVAAINTHDSI | 132 |
| | TYYPDSVEGRFTISRDNAKRMVYLQMNSLRAEDTAVYYCVTVRYKVFSYWGQGTQVT | |
| | VSS | |
| MSLN-A02 | EVQLVESGGGLVQPGGSLRLSCAASGFIFSFYFMGWFRQAPGKGRELVAAISRTGGS | 133 |
| | TYYPDSVEGRFTISRDNAKRMVYLQMNSLRAEDTAVYYCVVFHNWVGFGYWGQGTQV | |
| | TVSS | |
| MSLN-H08 | EVQLVESGGGLVQPGGSLRLSCAASGFIYSFDYMGWFRQAPGKGRELVAAISRTGGS | 134 |
| | TYYPDSVEGRFTISRDNAKRMVYLQMNSLRAEDTAVYYCVRMRWQMFGYWGQGTQVT | |
| | VSS | |
| MSLN-A03 | EVQLVESGGGLVQPGGSLRLSCAASGFIFNSNLMGWFRQAPGKGRELVAAISRTGGS | 135 |
| | TYYPDSVEGRFTISRDNAKRMVYLQMNSLRAEDTAVYYCVIIDKFNYIGFGYWGQGT | |
| | QVTVSS | |
| MSLN-A05 | EVQLVESGGGLVQPGGSLRLSCAASGYISNSYVMGWFRQAPGKGRELVAAISRTGGS | 136 |
| | TYYPDSVEGRFTISRDNAKRMVYLQMNSLRAEDTAVYYCAMATPDVLDYWGQGTQVT | |
| | VSS | |
| MSLN-F01 | EVQLVESGGGLVQPGGSLRLSCAASGSILNLDVMGWFRQAPGKGRELVAAISRTGGS | 137 |
| | TYYPDSVEGRFTISRDNAKRMVYLQMNSLRAEDTAVYYCARHTYISFGYWGQGTQVT | |
| | VSS | |
| MSLN-G01 | EVQLVESGGGLVQPGGSLRLSCAASGSIYNVNVMGWFRQAPGKGRELVAAISRTGGS | 138 |
| | TYYPDSVEGRFTISRDNAKRMVYLQMNSLRAEDTAVYYCVLTTPQSYGYWGQGTQVT | |
| | VSS | |
| MSLN-A09 | EVQLVESGGGLVQPGGSLRLSCAASGFIFGHNTMGWFRQAPGKGRELVAAISRTGGS | 139 |
| | TYYPDSVEGRFTISRDNAKRMVYLQMNSLRAEDTAVYYCALTRSDRPFTGSYRPEYS | |
| | FDYWGQGTQVTVSS | |
| MSLN-H06 | EVQLVESGGGLVQPGGSLRLSCAASGFIFNFDYMGWFRQAPGKGRELVAAINAYSDS | 140 |
| | TYYPDSVEGRFTISRDNAKRMVYLQMNSLRAEDTAVYYCVKTSYKIYGYWGQGTQVT | |
| | VSS | |
| H004-11- | EVQLVESGGGLVQPGGSLRLSCAASGFIYSFDYMGWFRQAPGKGRELVAAISRTGGS | 141 |
| 41BB | TYYPDSVEGRFTISRDNAKRMVYLQMNSLRAEDTAVYYCAVRMRWQMFGYWGQGTQV | |
| | TVSS | |
| H004-16- | EVQLVESGGGLVQPGGSLRLSCAASGFIFGHNTMGWFRQAPGKGRELVAAISRTGGS | 142 |
| 41BB | TYYPDSVEGRETISRDNAKRMVYLQMNSLRAEDTAVYYCALTRSDRPFTGSYRPEYS | |
| | FDYWGQGTQVTVSS | |
| H004-3- | EVQLVESGGGLVQPGGSLRLSCAASGSISGFYA MGWFRQAPGKGRELVAASINSED | 143 |
| 41BB | SITYYPDSVEGRFTISRDNAKRMVYLQMNSLRAEDTAVYYCVREKGKGFSYWGQGTQ | |
| | VTVSS | |
| H004-4- | EVQLVESGGGLVQPGGSLRLSCAASGSIFGFYAMGWFRQAPGKGRELVAAINSFDGI | 144 |
| 41BB | TYYPDSVEGRFTISRDNAKRMVYLQMNSLRAEDTAVYYCVRFRHMGFNYWGQGTQVT | |
| | VSS | |
| H004BC2-1 | EVQLVESGGGLVQPGGSLRLSCAASGSIYNAYAMGWFRQAPGKGRELVATINRIDST | 145 |
| | TYYPDSVEGRFTISRDNAKRMVYLQMNSLRAEDTAVYYCAARYIHAGFDYWGQGTQV | |
| | TVSS | |
| H004BC2-2 | EVQLVESGGGLVQPGGSLRLSCAASGSTFSYYAMGWFRQAPGKGRELVATINAFSDI | 146 |
| | TYYPDSVEGRFTISRDNAKRMVYLQMNSLRAEDTAVYYCAVREIYRSFGYWGQGTQV | |
| | TVSS | |
| H004BC2-3 | EVQLVESGGGLVQPGGSLRLSCAASGFISSFYAMGWFRQAPGKGRELVATINTESDI | 147 |
| | TYYPDSVEGRFTISRDNAKRMVYLQMNSLRAEDTAVYYCAVRLYREAFSYWGQGTQV | |
| | TVSS | |
| H004BC2-4 | EVQLVESGGGLVQPGGSLRLSCAASGYIYGFYAMGWFRQAPGKGRELVASINSIDSI | 148 |
| | TYYPDSVEGRFTISRDNAKRMVYLQMNSLRAEDTAVYYCAARYIAAGFGYWGQGTQV | |
| | TVSS | |
| H004BC2-5 | EVQLVESGGGLVQPGGSLRLSCAASGGIFDFYAMGWFRQAPGKGRELVASINSEDDI | 149 |
| | TYYPDSVEGRFTISRDNAKRMVYLQMNSLRAEDTAVYYCAARYYRVGFNYWGQGTQV | |
| | TVSS | |
| H004BC2-6 | EVQLVESGGGLVQPGGSLRLSCAASGSISSIYAMGWFRQAPGKGRELVATINTVDDI | 150 |
| | TYYPDSVEGRETISRDNAKRMVYLQMNSLRAEDTAVYYCAARYMTQSFSYWGQGTQV | |
| | TVSS | |
| H004BC2-7 | EVQLVESGGGLVQPGGSLRLSCAASGYTSNFDVMGWFRQAPGKGRELVAAINSIDDI | 151 |
| | TYYPDSVEGRFTISRDNAKRMVYLQMNSLRAEDTAVYYCAATYKWTLFSYWGQGTQV | |
| | TVSS | |
| H004BC2-8 | EVQLVESGGGLVQPGGSLRLSCAASGSISDLYAMGWFRQAPGKGRELVASINSFSDI | 152 |
| | TYYPDSVEGRFTISRDNAKRMVYLQMNSLRAEDTAVYYCAARMIKKGFDYWGQGTQV | |
| | TVSS | |
| H004BC2-9 | EVQLVESGGGLVQPGGSLRLSCAASGFISNFYSMGWFRQAPGKGRELVASINSFDSI | 153 |
| | TYYPDSVEGRFTISRDNAKRMVYLQMNSLRAEDTAVYYCAARLVRQAFNYWGQGTQV | |
| | TVSS | |
| H004BC2-10 | EVQLVESGGGLVQPGGSLRLSCAASGGTLRFYAMGWFRQAPGKGRELVATINSYDDI | 154 |
| | TYYPDSVEGRFTISRDNAKRMVYLQMNSLRAEDTAVYYCAARYAFQGENYWGQGTQV | |
| | TVSS | |
| H004BC2-11 | EVQLVESGGGLVQPGGSLRLSCAASGGTLRFYAMGWFRQAPGKGRELVATINSYDDI | 155 |
| | TYYPDSVEGRFTISRDNAKRMVYLQMNSLRAEDTAVYYCAVRYAFQGENYWGQGTQV | |
| | TVSS | |
| H004BC2-12 | EVQLVESGGGLVQPGGSLRLSCAASGFTSGIYAMGWFRQAPGKGRELVATINSESDI | 156 |
| | TYYPDSVEGRFTISRDNAKRMVYLQMNSLRAEDTAVYYCAARIFMKGENYWGQGTQV | |
| | TVSS | |
| H004BC2-13 | EVQLVESGGGLVQPGGSLRLSCAASGFISNFYAMGWFRQAPGKGRELVASINSYSDI | 157 |
| | TYYPDSVEGRFTISRDNAKRMVYLQMNSLRAEDTAVYYCAARWQGQGFGYWGQGTQV | |
| | TVSS | |
| H004BC2-14 | EVQLVESGGGLVQPGGSLRLSCAASGFTYDAYAMGWFRQAPGKGRELVATINTEDSN | 158 |
| | TYYPDSVEGRFTISRDNAKRMVYLQMNSLRAEDTAVYYCAARHMGMAFSYWGQGTQV | |
| | TVSS | |
| H004BC2-15 | EVQLVESGGGLVQPGGSLRLSCAASGSTSNYYAMGWFRQAPGKGRELVASINSEDSI | 159 |
| | TYYPDSVEGRFTISRDNAKRMVYLQMNSLRAEDTAVYYCAARKKGLAFSYWGQGTQV | |
| | TVSS | |
| H004BC2-16 | EVQLVESGGGLVQPGGSLRLSCAASGVTFNFYAMGWFRQAPGKGRELVATINSYNDI | 160 |
| | TYYPDSVEGRFTISRDNAKRMVYLQMNSLRAEDTAVYYCAARYAFQGENYWGQGTQV | |
| | TVSS | |
| H004BC2-17 | EVQLVESGGGLVQPGGSLRLSCAASGSTYGLYAMGWFRQAPGKGRELVASINSESDI | 161 |
| | TYYPDSVEGRFTISRDNAKRMVYLQMNSLRAEDTAVYYCAVRYYSKGFNYWGQGTQV | |
| | TVSS | |
| H004BC2-18 | EVQLVESGGGLVQPGGSLRLSCAASGSIYSIYAMGWFRQAPGKGRELVATINSYSDI | 162 |
| | TYYPDSVEGRETISRDNAKRMVYLQMNSLRAEDTAVYYCAARFYQVGENYWGQGTQV | |
| | TVSS | |
| H004BC2-19 | EVQLVESGGGLVQPGGSLRLSCAASGVTLGFYAMGWFRQAPGKGRELVATINSYDDI | 163 |
| | TYYPDSVEGRFTISRDNAKRMVYLQMNSLRAEDTAVYYCAARFSLQGENYWGQGTQV | |
| | TVSS | |
| H004BC2-20 | EVQLVESGGGLVQPGGSLRLSCAASGFTSNFYSMGWFRQAPGKGRELVASINSEDSI | 164 |
| | TYYPDSVEGRETISRDNAKRMVYLQMNSLRAEDTAVYYCAARKKGLAFSYWGQGTQV | |
| | TVSS | |

What is claimed is:

1. A mesothelin (MSLN) binding polypeptide comprising an immunoglobulin heavy chain variable domain comprising a CDR-H1, CDR-H2, and CDR-H3, wherein:
the CDR-H1 comprises a sequence having at least 90%, 95%, 99%, or 100% sequence identity to a sequence selected from SEQ ID NOs: 1-41;
the CDR-H2 comprises a sequence having at least 90%, 95%, 99%, or 100% sequence identity to a sequence selected from SEQ ID NOs: 42-82; and
the CDR-H3 comprises a sequence having at least 90%, 95%, 99%, or 100% sequence identity to a sequence selected from SEQ ID NOs: 83-123.

2. The MSLN binding polypeptide of claim 1, wherein:
1) the CDR-H1 comprises the sequence of SEQ ID NO: 1, the CDR-H2 comprises the sequence of SEQ ID NO: 42, and the CDR-H3 comprises the sequence of SEQ ID NO: 83;
2) the CDR-H1 comprises the sequence of SEQ ID NO: 2, the CDR-H2 comprises the sequence of SEQ ID NO: 43, and the CDR-H3 comprises the sequence of SEQ ID NO: 84;
3) the CDR-H1 comprises the sequence of SEQ ID NO: 3, the CDR-H2 comprises the sequence of SEQ ID NO: 44, and the CDR-H3 comprises the sequence of SEQ ID NO: 85;
4) the CDR-H1 comprises the sequence of SEQ ID NO: 4, the CDR-H2 comprises the sequence of SEQ ID NO: 45, and the CDR-H3 comprises the sequence of SEQ ID NO: 86;
5) the CDR-H1 comprises the sequence of SEQ ID NO: 5, the CDR-H2 comprises the sequence of SEQ ID NO: 46, and the CDR-H3 comprises the sequence of SEQ ID NO: 87;
6) the CDR-H1 comprises the sequence of SEQ ID NO: 6, the CDR-H2 comprises the sequence of SEQ ID NO: 47, and the CDR-H3 comprises the sequence of SEQ ID NO: 88;
7) the CDR-H1 comprises the sequence of SEQ ID NO: 7, the CDR-H2 comprises the sequence of SEQ ID NO: 48, and the CDR-H3 comprises the sequence of SEQ ID NO: 89;
8) the CDR-H1 comprises the sequence of SEQ ID NO: 8, the CDR-H2 comprises the sequence of SEQ ID NO: 49, and the CDR-H3 comprises the sequence of SEQ ID NO: 90;
9) the CDR-H1 comprises the sequence of SEQ ID NO: 9, the CDR-H2 comprises the sequence of SEQ ID NO: 50, and the CDR-H3 comprises the sequence of SEQ ID NO: 91;
10) the CDR-H1 comprises the sequence of SEQ ID NO: 10, the CDR-H2 comprises the sequence of SEQ ID NO: 51, and the CDR-H3 comprises the sequence of SEQ ID NO: 92;
11) the CDR-H1 comprises the sequence of SEQ ID NO: 11, the CDR-H2 comprises the sequence of SEQ ID NO: 52, and the CDR-H3 comprises the sequence of SEQ ID NO: 93;
12) the CDR-H1 comprises the sequence of SEQ ID NO: 12, the CDR-H2 comprises the sequence of SEQ ID NO: 53, and the CDR-H3 comprises the sequence of SEQ ID NO: 94;
13) the CDR-H1 comprises the sequence of SEQ ID NO: 13, the CDR-H2 comprises the sequence of SEQ ID NO: 54, and the CDR-H3 comprises the sequence of SEQ ID NO: 95;
14) the CDR-H1 comprises the sequence of SEQ ID NO: 14, the CDR-H2 comprises the sequence of SEQ ID NO: 55, and the CDR-H3 comprises the sequence of SEQ ID NO: 96;
15) the CDR-H1 comprises the sequence of SEQ ID NO: 15, the CDR-H2 comprises the sequence of SEQ ID NO: 56, and the CDR-H3 comprises the sequence of SEQ ID NO: 97;
16) the CDR-H1 comprises the sequence of SEQ ID NO: 16, the CDR-H2 comprises the sequence of SEQ ID NO: 57, and the CDR-H3 comprises the sequence of SEQ ID NO: 98;
17) the CDR-H1 comprises the sequence of SEQ ID NO: 17, the CDR-H2 comprises the sequence of SEQ ID NO: 58, and the CDR-H3 comprises the sequence of SEQ ID NO: 99;
18) the CDR-H1 comprises the sequence of SEQ ID NO: 18, the CDR-H2 comprises the sequence of SEQ ID NO: 59, and the CDR-H3 comprises the sequence of SEQ ID NO: 100;
19) the CDR-H1 comprises the sequence of SEQ ID NO: 19, the CDR-H2 comprises the sequence of SEQ ID NO: 60, and the CDR-H3 comprises the sequence of SEQ ID NO: 101;
20) the CDR-H1 comprises the sequence of SEQ ID NO: 20, the CDR-H2 comprises the sequence of SEQ ID NO: 61, and the CDR-H3 comprises the sequence of SEQ ID NO: 102;
21) the CDR-H1 comprises the sequence of SEQ ID NO: 21, the CDR-H2 comprises the sequence of SEQ ID NO: 62, and the CDR-H3 comprises the sequence of SEQ ID NO: 103;
22) the CDR-H1 comprises the sequence of SEQ ID NO: 22, the CDR-H2 comprises the sequence of SEQ ID NO: 63, and the CDR-H3 comprises the sequence of SEQ ID NO: 104;
23) the CDR-H1 comprises the sequence of SEQ ID NO: 23, the CDR-H2 comprises the sequence of SEQ ID NO: 64, and the CDR-H3 comprises the sequence of SEQ ID NO: 105;
24) the CDR-H1 comprises the sequence of SEQ ID NO: 24, the CDR-H2 comprises the sequence of SEQ ID NO: 65, and the CDR-H3 comprises the sequence of SEQ ID NO: 106;
25) the CDR-H1 comprises the sequence of SEQ ID NO: 25, the CDR-H2 comprises the sequence of SEQ ID NO: 66, and the CDR-H3 comprises the sequence of SEQ ID NO: 107;
26) the CDR-H1 comprises the sequence of SEQ ID NO: 26, the CDR-H2 comprises the sequence of SEQ ID NO: 67, and the CDR-H3 comprises the sequence of SEQ ID NO: 108;
27) the CDR-H1 comprises the sequence of SEQ ID NO: 27, the CDR-H2 comprises the sequence of SEQ ID NO: 68, and the CDR-H3 comprises the sequence of SEQ ID NO: 109;
28) the CDR-H1 comprises the sequence of SEQ ID NO: 28, the CDR-H2 comprises the sequence of SEQ ID NO: 69, and the CDR-H3 comprises the sequence of SEQ ID NO: 110;
29) the CDR-H1 comprises the sequence of SEQ ID NO: 29, the CDR-H2 comprises the sequence of SEQ ID NO: 70, and the CDR-H3 comprises the sequence of SEQ ID NO: 111;
30) the CDR-H1 comprises the sequence of SEQ ID NO: 30, the CDR-H2 comprises the sequence of SEQ ID NO: 71, and the CDR-H3 comprises the sequence of SEQ ID NO: 112;
31) the CDR-H1 comprises the sequence of SEQ ID NO: 31, the CDR-H2 comprises the sequence of SEQ ID NO: 72, and the CDR-H3 comprises the sequence of SEQ ID NO: 113;
32) the CDR-H1 comprises the sequence of SEQ ID NO: 32, the CDR-H2 comprises the sequence of SEQ ID NO: 73, and the CDR-H3 comprises the sequence of SEQ ID NO: 114;
33) the CDR-H1 comprises the sequence of SEQ ID NO: 33, the CDR-H2 comprises the sequence of SEQ ID NO: 74, and the CDR-H3 comprises the sequence of SEQ ID NO: 115;
34) the CDR-H1 comprises the sequence of SEQ ID NO: 34, the CDR-H2 comprises the sequence of SEQ ID NO: 75, and the CDR-H3 comprises the sequence of SEQ ID NO: 116;
35) the CDR-H1 comprises the sequence of SEQ ID NO: 35, the CDR-H2 comprises the sequence of SEQ ID NO: 76, and the CDR-H3 comprises the sequence of SEQ ID NO: 117;
36) the CDR-H1 comprises the sequence of SEQ ID NO: 36, the CDR-H2 comprises the sequence of SEQ ID NO: 77, and the CDR-H3 comprises the sequence of SEQ ID NO: 118;
37) the CDR-H1 comprises the sequence of SEQ ID NO: 37, the CDR-H2 comprises the sequence of SEQ ID NO: 78, and the CDR-H3 comprises the sequence of SEQ ID NO: 119;
38) the CDR-H1 comprises the sequence of SEQ ID NO: 38, the CDR-H2 comprises the sequence of SEQ ID NO: 79, and the CDR-H3 comprises the sequence of SEQ ID NO: 120;
39) the CDR-H1 comprises the sequence of SEQ ID NO: 39, the CDR-H2 comprises the sequence of SEQ ID NO: 80, and the CDR-H3 comprises the sequence of SEQ ID NO: 121;
40) the CDR-H1 comprises the sequence of SEQ ID NO: 40, the CDR-H2 comprises the sequence of SEQ ID NO: 81, and the CDR-H3 comprises the sequence of SEQ ID NO: 122; or
41) the CDR-H1 comprises the sequence of SEQ ID NO: 41, the CDR-H2 comprises the sequence of SEQ ID NO: 82, and the CDR-H3 comprises the sequence of SEQ ID NO: 123.

3. The MSLN binding polypeptide of claim 1, wherein the heavy chain variable domain comprises an amino acid sequence having at least 90%, 95%, 99%, or 100% sequence identity to any sequence selected from SEQ ID NOs: 124-164.

4. The MSLN binding polypeptide of claim 1, wherein the MSLN binding polypeptide is humanized.

5. The MSLN binding polypeptide of claim 1, wherein the MSLN binding polypeptide is a single domain antibody (sdAb).

6. A chimeric antigen receptor (CAR) comprising the MSLN binding polypeptide of claim 1.

7. A chimeric antigen receptor (CAR) cell comprising the CAR of claim 6.

8. The CAR cell of claim 7, wherein the CAR cell is a CART cell.

9. The CAR cell of claim 7, wherein the CAR cell comprises at least two binding polypeptides and the CAR cell is a multivalent CAR cell.

10. The CAR cell of claim 7, wherein the CAR cell is derived from a subject or from a cell line.

11. The CAR cell of claim 10, wherein the subject has a cancer.

12. The CAR cell of claim 11, wherein the cancer is breast cancer, colorectal cancer, kidney cancer, liver cancer, lung cancer, brain cancer, pancreatic cancer, bladder cancer, testicular cancer, prostate cancer, gastric cancer, ovarian cancer, head and neck cancer, gallbladder cancer, a hematologic malignancy, or any combination thereof.

13. A nucleic acid that encodes for a polypeptide comprising a sequence having at least 90% sequence identity to the MSLN binding polypeptide of claim 1.

14. A method of treating a cancer in a subject in need thereof, comprising administering the CAR cell of claim 7.

15. The method of claim 14, wherein the chimeric antigen receptor cell is autologous or allogeneic to the subject.

16. The method of claim 14, wherein the subject is a mammal.

17. The method of claim 14, wherein the subject is a human.

18. The method of claim 14, wherein the cancer is breast cancer, colorectal cancer, kidney cancer, liver cancer, lung cancer, brain cancer, pancreatic cancer, bladder cancer, testicular cancer, prostate cancer, gastric cancer, ovarian cancer, head and neck cancer, gallbladder cancer, a hematologic malignancy, or any combination thereof.

19. The method of claim 14, wherein the chimeric antigen receptor cell is administered parenterally.
